SEQUENCE LISTING (223 sequences)

SEQ ID NO: 1 - protein, 120 aa, synthetic construct
QVQLQESGPGLVKPSGTLSLTCAVSGGSISSSNWWSWVRQPPGKGLEWIGEIYHSGNTNYNPSLKSRVTVSVDKSKNQFSLKLTSVTAADTAVYYCARDVSGGVNWFDPWGQGTLVTVSS

SEQ ID NO: 2 - protein, 110 aa, synthetic construct
NFMLTQPHSVSESPGKTVTISCTRSSGSIASNYVQWYQQRPGSAPTTVIYEDNQRPSGVPDRFSGSIDSSSNSASLTISGLKTEDEADYYCQSYDSSTVVFGGGTKLTVL

SEQ ID NO: 3 - protein, 121 aa, synthetic construct
EVQLVQSGAEVKKPGESLKISCKGSGYTYTSYWMNWVRQMPGKGLEWMGRIDPYDSETHYDQKFKDQVTLSVDKSISTAYLQWSSLKASDTAMYYCAGGITTILGGYFDYWGQGTLVTVSS

SEQ ID NO: 4 - protein, 109 aa, synthetic construct
DIQMTQSPSSLSASVGDRVTITCRASQDISNYLNWYQQKPGKAPKLLIYYTSRLHSGVPSRFSGSGSGTDYTLTISSLQPEDFATYYCQQGNTLPPYTFGQGTKVEIKR

SEQ ID NO: 5 - protein, 122 aa, synthetic construct
EVQLVESGGGLVQPGGSLRLSCKASGYAYTSYWMNWVRQAPGKGLVWVGRIDPYDSETHYNQKFKDRFTLSVDKAKSTTYLQMNSLRAEDTAVYYCATLITTLRGEGAMEYWGQGTLVTVSS

SEQ ID NO: 6 - protein, 108 aa, synthetic construct
DLQMTQSPSSLSASVGDRVTITCKASQDVNTAVAWYQQKPGKAPKLLIYWASTRHTGVPSRFSGSGSGTDYTLTISSLQPEDFATYYCQQHYSTPFTFGQGTKVEIKR

SEQ ID NO: 7 - protein, 119 aa, synthetic construct
QVQLQESGPGLVKPSETLSLTCSVTGYSITSDYYWHWIRQPPGKGLEWIAYIRYDGRNDYNPSLKNRVTISRDTSKNQFSLKLSSVTAADTAVYYCAREDYGSSSFDYWGQGTLVTVSS

SEQ ID NO: 8 - protein, 108 aa, synthetic construct
DIQMTQSPSSLSASVGDRVTITCRASENIYSNLAWYQQKPGKAPKLLVYAATNLADGVPSRFSGSGSGTDYTLTISSLQPEDFATYYCQHFWDSPPTFGQGTKVEIKR

SEQ ID NO: 9 - protein, 119 aa, synthetic construct
QVQLQESGPGLVKPSQTLSLTCSVTGYSITSDYYWHWIRQPPGKGLEWIAYIRYDGRNDSNPSLKNRVTISRDTSKNQFSLKLSSVTAADTAVYYCAREDYASSSFDYWGQGTLVTVSS

SEQ ID NO: 10 - protein, 108 aa, synthetic construct
DIQMTQSPSSLSASVGDRVTITCRPSENIYSNLAWYQQKPGKAPKLLVYAATNLADGVPSRFSGSGSGTDYTLTISSLQPEDIATYYCQHFWDSPPTFGQGTKLEIKR

SEQ ID NO: 11 - protein, 9 aa, synthetic construct
GGSISSSNW

SEQ ID NO: 12 - protein, 7 aa, synthetic construct
YHSGNTN

SEQ ID NO: 13 - protein, 13 aa, synthetic construct
ARDVSGGVNWFDP

SEQ ID NO: 14 - protein, 8 aa, synthetic construct
SGSIASNY

SEQ ID NO: 15 - skipped

SEQ ID NO: 16 - protein, 9 aa, synthetic construct
QSYDSSTVV

SEQ ID NO: 17 - protein, 5 aa, synthetic construct
SYWMN

SEQ ID NO: 18 - protein, 17 aa, synthetic construct
RIDPYDSETHYDQKFKD

SEQ ID NO: 19 - protein, 12 aa, synthetic construct
GITTILGGYFDY

SEQ ID NO: 20 - protein, 11 aa, synthetic construct
RASQDISNYLN

SEQ ID NO: 21 - protein, 7 aa, synthetic construct
YTSRLHS

SEQ ID NO: 22 - protein, 10 aa, synthetic construct
QQGNTLPPYT

SEQ ID NO: 23 - protein, 5 aa, synthetic construct
SYWMN

SEQ ID NO: 24 - protein, 17 aa, synthetic construct
RIDPYDSETHYNQKFKD

SEQ ID NO: 25 - protein, 13 aa, synthetic construct
LITTLRGEGAMEY

SEQ ID NO: 26 - protein, 11 aa, synthetic construct
KASQDVNTAVA

SEQ ID NO: 27 - protein, 7 aa, synthetic construct
WASTRHT

SEQ ID NO: 28 - protein, 9 aa, synthetic construct
QQHYSTPFT

SEQ ID NO: 29 - protein, 6 aa, synthetic construct
SDYYWH

SEQ ID NO: 30 - protein, 21 aa, synthetic construct
YIRYDGRNDYNPSLKNRVTIS

SEQ ID NO: 31 - protein, 10 aa, synthetic construct
SSFDYWGQGT

SEQ ID NO: 32 - protein, 11 aa, synthetic construct
RASENIYSNLA

SEQ ID NO: 33 - protein, 7 aa, synthetic construct
AATNLAD

SEQ ID NO: 34 - protein, 9 aa, synthetic construct
QHFWDSPPT

SEQ ID NO: 35 - protein, 6 aa, synthetic construct
SDYYWH

SEQ ID NO: 36 - protein, 16 aa, synthetic construct
YIRYDGRNDSNPSLKN

SEQ ID NO: 37 - protein, 10 aa, synthetic construct
EDYASSSFDY

SEQ ID NO: 38 - protein, 11 aa, synthetic construct
RPSENIYSNLA

SEQ ID NO: 39 - protein, 7 aa, synthetic construct
AATNLAD

SEQ ID NO: 40 - protein, 9 aa, synthetic construct
QHFWDSPPT

SEQ ID NO: 41 - protein, 7 aa, synthetic construct
LQNRKLV

SEQ ID NO: 42 - DNA, 360 bp, synthetic construct
  source 1..360: mol_type = other DNA
caggtccagctgcaagagtctggccctggactggtcaagccttctggcaccctgtctctgacatgtgctgtgtccggcggctccatctcctcctctaattggtggtcttgggtccgacagcctcctggcaaaggactggaatggatcggcgagatctaccactccggcaacaccaactacaaccccagcctgaagtccagagtgaccgtgtccgtggacaagtccaagaaccagttctccctgaagctgacctctgtgaccgctgccgataccgccgtgtactactgtgctagagatgtgtctggcggagtgaattggttcgatccttggggccagggcacactggttaccgtgtcctct

SEQ ID NO: 43 - DNA, 330 bp, synthetic construct
  source 1..330: mol_type = other DNA
aactttatgctgacccagcctcactccgtgtccgagtctccaggcaagaccgtgaccatctcctgtaccagatcctccggctctatcgcctccaactacgtgcagtggtatcagcagaggcctggctctgctcctaccaccgtgatctacgaggacaaccagaggccttctggcgtgcccgataggttctctggctccatcgactcctcttccaactccgcctctctgaccatcagcggcctgaaaaccgaggacgaggccgactactactgccagtcctacgactcttccaccgtggtgtttggcggcggaacaaagctgacagtgctg

SEQ ID NO: 44 - DNA, 363 bp, synthetic construct
  source 1..363: mol_type = other DNA
gaagttcagctggttcagtctggcgccgaagtgaagaagcctggcgagtccctgaagatctcctgcaaaggctccggctacacctacaccagctactggatgaactgggtccgacagatgcctggcaaaggcctggaatggatgggcagaatcgacccctacgactccgagacacactacgaccagaaattcaaggaccaagtgaccctgagcgtggacaagtccatctccaccgcttacctgcagtggtcctctctgaaggcctctgacaccgccatgtactactgtgctggcggcatcaccaccatcctcggcggctactttgattactggggccagggcacactggtcaccgtttcttcc

SEQ ID NO: 45 - DNA, 327 bp, synthetic construct
  source 1..327: mol_type = other DNA
gacatccagatgacccagtctccatcctctctgtccgcctctgtgggcgacagagtgaccatcacctgtagagccagccaggacatctccaactacctgaactggtatcagcagaagcccggcaaggcccctaagctgctgatctactacacctctcggctgcactctggcgtgccctctagattttctggctccggctctggcaccgactataccctgacaatctccagcctgcagcctgaggacttcgccacctactattgccagcagggcaacaccctgcctccatacacatttggccagggcaccaaggtggaaatcaagcgt

SEQ ID NO: 46 - DNA, 366 bp, synthetic construct
  source 1..366: mol_type = other DNA
gaagtgcagctggttgaatctggcggcggattggttcagcctggcggatctctgagactgtcctgcaaggcttctggctacgcctacacctcctactggatgaactgggtccgacaggctcctggcaaaggactcgtgtgggtcggaagaatcgacccctacgactccgagacacactacaaccagaagttcaaggaccggttcaccctgagcgtggacaaggccaagtctaccacctacctgcagatgaactccctgagagccgaggacaccgccgtgtactattgcgctaccctgatcaccacactgagaggcgagggcgccatggaatattggggacagggaaccctggtcaccgtgtcctct

SEQ ID NO: 47 - DNA, 324 bp, synthetic construct
  source 1..324: mol_type = other DNA
gacctgcagatgacccagtctccttccagcctgtctgcctctgtgggcgacagagtgaccatcacatgcaaggccagccaggacgtgaacaccgccgttgcttggtatcagcagaagcctggcaaggcccctaagctgctgatctactgggcctccaccagacataccggcgtgccctctagattctccggctctggctctggcaccgactataccctgacaatctccagcctgcagcctgaggacttcgccacctactactgccagcagcactacagcacccctttcacctttggccagggcaccaaggtggaaatcaagcgt

SEQ ID NO: 48 - DNA, 357 bp, synthetic construct
  source 1..357: mol_type = other DNA
caggttcagctgcaagagtctggccctggcctggtcaagccttccgaaacactgtccctgacctgcagcgtgaccggctactctatcacctccgactactactggcactggatcagacagcctccaggcaaaggcctggaatggatcgcctacatcagatacgacggccggaacgactacaaccccagcctgaagaacagagtgaccatcagccgggacacctccaagaaccagttctccctgaagctgtcctccgtgaccgctgctgataccgccgtgtactactgcgccagagaggactacggctcctcctcctttgattactggggccagggcaccctggtcaccgttagttct

SEQ ID NO: 49 - DNA, 324 bp, synthetic construct
  source 1..324: mol_type = other DNA
gacatccagatgacccagtctccatcctctctgtccgcctctgtgggcgacagagtgaccatcacctgtcgggcctccgagaacatctactccaacctggcctggtatcagcagaagcctggcaaggctcctaagctgctggtgtacgccgctaccaatctggctgatggcgtgccctctagattctccggctctggctctggcaccgactataccctgacaatctccagcctgcagcctgaggacttcgccacctactactgccagcacttctgggacagccctccaacctttggccagggcaccaaggtggaaatcaagcgt

SEQ ID NO: 50 - DNA, 357 bp, synthetic construct
  source 1..357: mol_type = other DNA
caggttcagctgcaagagtctggccctggcctggtcaagccttctcagaccctgtctctgacctgcagcgtgaccggctactccatcacctccgactactactggcactggatcagacagcctccaggcaaaggcctggaatggatcgcctacatcagatacgacggccggaacgacagcaaccccagcctgaagaacagagtgaccatcagccgggacacctccaagaaccagttctccctgaagctgtcctccgtgaccgctgctgataccgccgtgtactactgcgccagagaggactacgcctcctcctcctttgattactggggccagggcaccctggtcaccgttagttct

SEQ ID NO: 51 - DNA, 324 bp, synthetic construct
  source 1..324: mol_type = other DNA
gacatccagatgacccagtctccatcctctctgtccgcctctgtgggcgacagagtgaccatcacctgtcggccttccgagaacatctactccaacctggcctggtatcagcagaagcctggcaaggctcctaagctgctggtgtacgccgctaccaatctggctgatggcgtgccctctagattctccggctctggctctggcaccgactataccctgacaatctccagcctgcagcctgaggatatcgccacctactactgccagcacttctgggacagccctccaacctttggccagggcaccaagctggaaatcaagcgt

SEQ ID NO: 52 - protein, 1357 aa, Toxoplasma gondii
MLASPQGDPETGVSPVSDGAWRASVGGRPRPDTSDRGESDAGCTARTATSPPPRSLPRIAVVVRKRPLNEAERKRNEADLVQTRGRNAVLVDEPREKVDLTPYVMRHEFRVDFAFDEKSTNDEVYRAVVRPLVEACCLGDANTSCFAYGQTGSGKTYTMLGPQPYGRGVEAGVFELAAEDIFKCLEGGEKDAFVSFFEIYNGKLFDLLQNRKLVAALENGKKEVVVRDLRMEQVRDKEMLLSKMIEGIELRKIGVNSVNDESSRSHAILQVIFRKRNSGEACGRIAFIDLAGSERGADTLQHSRQTQQDGAGINRSLLALKECIRAMDQDKGHIPFRDSELTKVLREIFVGRSSRSVMIATVSPSTSCCEQTLNTLRYASRVKNFRQTPPQPTAVVPASSPLPPRPSSSTAESSSLAFSRSISSANAAARQARASLPCMHAASSDGLGTLAEKLPKSGFDEPHLGALGEFPTPHLAEATHALSPLESGSPSRVSAKGARAKGEGLGRAGSAAGPRAQGSTRSRARPETVPARSSLEKLHEEASSSSDARAETDAEMHASGPLRLAAGLGPGVAAGADVQRDRSSLSASTCAPSSCERLSSCGSAQTAQRRAASVRPSRPATSQASPQLERRGGEPKSAEDAEPSGERDKRYRGASSGGSVARPVTGPGPSLGMGRPLTRLQASSGSSEEFRSSQDLDAEEGEMENGPAFLATRSTSRKLATGSRAAPERASLLPRLRGTRAACASPFGASRTSLGAASPVPNLPDSASPDVETLGFGSEPKTTRGRGGEAFEISLGSDGGRKKGALRAALPKAEESSSEATPRSEGLSATRLYKTTRLLGKPVARSRGLRSATAVSEPNAQGVREKRSSDVEKGDSSSSEKDASAPGKARGGGRPGAASTPPLSLLRKSWLRANHEQQAQDGALVCPDGSSIIFVPRHGRAREASPLSSREKKPTATALARSVGPNASPEDAEMAERADGGLGLSKKRREDEAEACGDARRDQRSEDAAPRGARVTVPSYLVDKEELRPASAAVPPRAAAFPSKLPRPSPGAHAPGLSLDAGLGGDREKIKSPVAVALDASSQRLLAHLLPRAGFRGATLASPRRAPEQAAAGERGEASGDAPALVKRKKQGEENARDACVAESAKGSAREERIHFGCGREPQTILQEAFACNADYHNLDLAELDRLQDAVADRRDTCMQQLLQLAKKRAEEASSRHGINESQQDILVCRLHQANPGSSEFGEYMRQRMEADFRKLLAMRQLWVEAEELRMLNRLLTSEYQTKSGAARAPAPGGALPEAGLSPSSRRLGAGDGDSASPSFWKKPDLETARDVEMTEAFSSPPLLSGCSPVAESGISA

SEQ ID NO: 53-99 - skipped (47 entries)

SEQ ID NO: 100 - protein, 10 aa, Toxoplasma gondii
LLQNRKLVAA

SEQ ID NO: 101 - protein, 10 aa, Toxoplasma gondii
DLLQNRKLVA

SEQ ID NO: 102 - protein, 9 aa, Toxoplasma gondii
LLQNRKLVA

SEQ ID NO: 103 - protein, 8 aa, Toxoplasma gondii
LQNRKLVA

SEQ ID NO: 104 - protein, 8 aa, Toxoplasma gondii
LLQNRKLV

SEQ ID NO: 105 - protein, 11 aa, unidentified
MNLQNRKLVQV

SEQ ID NO: 106 - protein, 7 aa, Toxoplasma gondii
LQNRKLV

SEQ ID NO: 107-199 - skipped (93 entries)

SEQ ID NO: 200 - protein, 23 aa, unidentified
KFANYSIMNLQNRKLVQVGIYNG

SEQ ID NO: 201 - protein, 13 aa, Toxoplasma gondii
NRKLVAALENGKK

SEQ ID NO: 202 - protein, 14 aa, Toxoplasma gondii
QNRKLVAALENGKK

SEQ ID NO: 203 - protein, 15 aa, Toxoplasma gondii
LQNRKLVAALENGKK

SEQ ID NO: 204 - protein, 15 aa, Toxoplasma gondii
LLQNRKLVAALENGK

SEQ ID NO: 205 - protein, 17 aa, Toxoplasma gondii
DLLQNRKLVAALENGKK

SEQ ID NO: 206 - protein, 18 aa, Toxoplasma gondii
FDLLQNRKLVAALENGKK

SEQ ID NO: 207 - protein, 19 aa, Toxoplasma gondii
LFDLLQNRKLVAALENGKK

SEQ ID NO: 208 - protein, 20 aa, Toxoplasma gondii
KLFDLLQNRKLVAALENGKK

SEQ ID NO: 209 - protein, 21 aa, Toxoplasma gondii
GKLFDLLQNRKLVAALENGKK

SEQ ID NO: 210 - protein, 22 aa, Toxoplasma gondii
NGKLFDLLQNRKLVAALENGKK

SEQ ID NO: 211 - protein, 23 aa, Toxoplasma gondii
YNGKLFDLLQNRKLVAALENGKK

SEQ ID NO: 212 - protein, 24 aa, Toxoplasma gondii
IYNGKLFDLLQNRKLVAALENGKK

SEQ ID NO: 213 - protein, 23 aa, Toxoplasma gondii
IYNGKLFDLLQNRKLVAALENGK

SEQ ID NO: 214 - protein, 22 aa, Toxoplasma gondii
IYNGKLFDLLQNRKLVAALENG

SEQ ID NO: 215 - protein, 20 aa, Toxoplasma gondii
IYNGKLFDLLQNRKLVAALE

SEQ ID NO: 216 - protein, 20 aa, Toxoplasma gondii
IYNGKLFDLLQNRKLVAALE

SEQ ID NO: 217 - protein, 19 aa, Toxoplasma gondii
IYNGKLFDLLQNRKLVAAL

SEQ ID NO: 218 - protein, 18 aa, Toxoplasma gondii
IYNGKLFDLLQNRKLVAA

SEQ ID NO: 219 - protein, 17 aa, Toxoplasma gondii
IYNGKLFDLLQNRKLVA

SEQ ID NO: 220 - protein, 16 aa, Toxoplasma gondii
IYNGKLFDLLQNRKLV

SEQ ID NO: 221 - protein, 15 aa, Toxoplasma gondii
IYNGKLFDLLQNRKL

SEQ ID NO: 222 - protein, 14 aa, Toxoplasma gondii
IYNGKLFDLLQNRK

SEQ ID NO: 223 - protein, 13 aa, Toxoplasma gondii
IYNGKLFDLLQNR

(19) United States
(12) Patent Application Publication
(10) Pub. No.: US 2025/0263488 A1
(43) Pub. Date: Aug. 21, 2025

(54) METHODS AND SYSTEMS FOR IDENTIFYING OR TREATING CENTRAL NERVOUS SYSTEMS DISEASE

(71) Applicant: ARIALYS THERAPEUTICS, INC., La Jolla, CA (US)

(72) Inventors: Mitsuyuki MATSUMOTO, San Diego, CA (US); Shanni YAMAKI, La Jolla, CA (US); Amir RAZAI, San Francisco, CA (US); Roghiye KAZIMI, Spring Valley, CA (US)

(21) Appl. No.: 19/035,338

(22) Filed: Jan. 23, 2025

Related U.S. Application Data
(63) Continuation of application No. PCT/US2023/071138, filed on Jul. 27, 2023.
(60) Provisional application No. 63/369,923, filed on Jul. 29, 2022.

(51) Int. Cl. C07K 16/28; A61K 39/00; A61P 25/08; A61P 25/18; A61P 25/24; A61P 25/28; G01N 33/564

(52) U.S. Cl. CPC C07K 16/286; A61P 25/08; A61P 25/18; A61P 25/24; A61P 25/28; G01N 33/564; A61K 2039/505; C07K 2317/33; C07K 2317/34; C07K 2317/77; G01N 2333/70571; G01N 2500/04; G01N 2800/2814; G01N 2800/2857; G01N 2800/30; G01N 2800/302; G01N 2800/304; G01N 2800/50; G01N 2800/7095

(57) ABSTRACT

Systems and methods for the identification of auto-antibodies associated with central nervous system or psychiatric disease or disorders. Also disclosed herein are methods of treating central nervous system or psychiatric disease or disorders associated with auto-antibodies.

### CROSS REFERENCE

This application is a continuation of International Application No. PCT/US2023/071138, filed Jul. 27, 2023, which claims the benefit of U.S. Provisional Application No. 63/369,923, filed on Jul. 29, 2022, each of which is incorporated by reference in its entirety.

### SEQUENCE LISTING

The instant application contains a Sequence Listing which has been submitted electronically in XML format and is hereby incorporated by reference in its entirety. Said XML copy, created on Feb. 19, 2025, is named 62548-704_301_SL.xml and is 106,038 bytes in size.

### BACKGROUND OF THE INVENTION

Autoimmunity is the result of the body producing T-cells and/or antibodies specific for self-antigens. This immune response can be triggered by environmental exposures including viruses, pathogenic organisms, or tumor growth. The immune responses can become chronic, leading to a variety of conditions including psychiatric and central nervous system (CNS) disorders or diseases caused by the production of autoantibodies. Viral, bacterial, and parasitic infections, especially Toxoplasmosis, are well known to increase a risk of schizophrenia and related psychiatric disorders. One potential mechanism for the infection-induced CNS diseases is molecular mimicry where pathogenic organism's proteins have similar sequence with proteins expressed in human brains and generate cross-reactive auto-antibodies. There is a need to identify auto-antibodies, associated antigenic peptides, and treatments for diseases associated with auto-antibodies.

### SUMMARY OF THE INVENTION

Disclosed herein is a method for the treatment or improvement of a psychiatric or central nervous system disease or disorder comprising administering to a subject in need thereof an antibody or antigen binding fragment thereof comprising a heavy chain variable region and a light chain variable region, the heavy and light chain variable region comprising: (a) a heavy chain CDR1 as set forth in SEQ ID NO: 11, a heavy chain CDR2 as set forth in SEQ ID NO: 12, a heavy chain CDR3 as set forth in SEQ ID NO: 13, a light chain CDR1 as set forth in SEQ ID NO: 14, a light chain CDR2 as set forth in SEQ ID NO:15, and/or a light chain CDR3 as set forth in SEQ ID NO:16; (b) a heavy chain CDR1 as set forth in SEQ ID NO: 17, a heavy chain CDR2 as set forth in SEQ ID NO: 18, a heavy chain CDR3 as set forth in SEQ ID NO: 19, a light chain CDR1 as set forth in SEQ ID NO: 20, a light chain CDR2 as set forth in SEQ ID NO:21, and/or a light chain CDR3 as set forth in SEQ ID NO:22; (c) a heavy chain CDR1 as set forth in SEQ ID NO:23, a heavy chain CDR2 as set forth in SEQ ID NO: 24, a heavy chain CDR3 as set forth in SEQ ID NO: 25, a light chain CDR1 as set forth in SEQ ID NO: 26, a light chain CDR2 as set forth in SEQ ID NO:27, and/or a light chain CDR3 as set forth in SEQ ID NO:28; (d) a heavy chain CDR1 as set forth in SEQ ID NO: 29, a heavy chain CDR2 as set forth in SEQ ID NO: 30, a heavy chain CDR3 as set forth in SEQ ID NO: 31, a light chain CDR1 as set forth in SEQ ID NO: 32, a light chain CDR2 as set forth in SEQ ID NO: 33, and/or a light chain CDR3 as set forth in SEQ ID NO:34; or (e) a heavy chain CDR1 as set forth in SEQ ID NO: 35, a heavy chain CDR2 as set forth in SEQ ID NO: 36, a heavy chain CDR3 as set forth in SEQ ID NO: 37, a light chain CDR1 as set forth in SEQ ID NO: 38, a light chain CDR2 as set forth in SEQ ID NO:39, and/or a light chain CDR3 as set forth in SEQ ID NO:40. The heavy chain variable region can comprise an amino acid sequence that has at least 80% sequence identity to any one of SEQ ID NOs: 1, 3, 5, 7, or 9, and the light chain variable region can comprise an amino acid sequence that has at least 80% sequence identity to any one of SEQ ID NOs: 2, 4, 6, 8, or 10. The heavy chain variable region can comprise an amino acid sequence that has at least 85% sequence identity to any one of SEQ ID NOs: 1, 3, 5, 7, or 9, and the light chain variable region can comprise an amino acid sequence that has at least 85% sequence identity to any one of SEQ ID NOs: 2, 4, 6, 8, or 10. The heavy chain variable region can comprise an amino acid sequence that has at least 90% sequence identity to any one of SEQ ID NOs: 1, 3, 5, 7, or 9, and the light chain variable region can comprise an amino acid sequence that has at least 90% sequence identity to any one of SEQ ID NOs: 2, 4, 6, 8, or 10. The heavy chain variable region can comprise an amino acid sequence that has at least 95% sequence identity to any one of SEQ ID NOs: 1, 3, 5, 7, or 9, and the light chain variable region can comprise an amino acid sequence that has at least 95% sequence identity to any one of SEQ ID NOs: 2, 4, 6, 8, or 10. The heavy chain variable region can comprise an amino acid sequence that has at least 98% sequence identity to any one of SEQ ID NOs: 1, 3, 5, 7, or 9, and the light chain variable region can comprise an amino acid sequence that has at least 98% sequence identity to any one of SEQ ID NOs: 2, 4, 6, 8, or 10. The heavy chain variable region can comprise an amino acid sequence identical to any one of SEQ ID NOs: 1, 3, 5, 7, or 9, and the light chain variable region can comprise an amino acid sequence identical to any one of SEQ ID NOs: 2, 4, 6, 8, or 10.

The psychiatric or central nervous system disease or disorder can be an autoantibody associated disorder. The psychiatric or central nervous system disease or disorder can be schizophrenia. The psychiatric or central nervous system disease or disorder can be psychosis, bipolar disorder, depression, epilepsy and dementia. In some embodiments, the psychiatric or central nervous system disease or disorder is not encephalitis.

The antibody or antigen binding fragment thereof can bind an antigen expressed by a neuron and/or glia of said subject. The antibody or antigen binding fragment thereof can bind to a N-methyl-D-aspartate (NMDA) receptor of the subject. The antibody or antigen binding fragment thereof can bind a NMDAR1 (NR1) subunit of the NMDA receptor. The antibody or antigen binding fragment thereof can bind an epitope of NR1 comprising amino acid sequence LQNRKLV (SEQ ID NO: 41). The antibody or antigen binding fragment thereof can bind at least one amino acid of NR1 consisting of amino acid sequence LQNRKLV (SEQ ID NO: 41).

Disclosed herein is a method for identifying an antibody or antigen binding fragment thereof for use in treatment of a psychiatric or central nervous system disease or disorder, the method comprising identifying said antibody or antigen binding fragment thereof having the following property: binds specifically to an epitope LQNRKLV (SEQ ID NO: 41) of a NR1 subunit of a NMDA receptor. The antibody or antigen binding fragment thereof can inhibit binding of an autoantibody that binds to said NMDA receptor. The antibody or antigen binding fragment thereof can bind to the epitope with a K_{D} lower than 1 mM. The antibody or antigen binding fragment thereof can comprise a human antibody. The antibody or antigen binding fragment thereof can be humanized.

Disclosed herein is a method for the treatment or improvement of a psychiatric or central nervous system disease or disorder comprising identifying a subject as having been exposed to a pathogenic organism, wherein said identifying comprises identifying an antibody that binds to an immunogenic epitope of said pathogenic organism. The method can further comprise identifying said subject as having one or more symptoms of a psychiatric or central nervous system disease or disorder. The method can further comprise outputting a report identifying said subject as being at high risk for N-methyl-D-aspartate (NMDA) receptor dysfunction. The psychiatric or central nervous system disease or disorder can be anti-NMDAR encephalitis, schizophrenia, psychosis, bipolar disorder, depression, epilepsy, or dementia. The psychiatric or central nervous system disease or disorder is not encephalitis. The pathogenic organism can be from the genus of Toxoplasma, Paramecium, Campylobacter, Enterococcus, Peptoniphilus, Paenalcaligenes, Pseudomonas, Burkholderia, Chromobacterium, Acinetobacter, Paenibacillus, Escherichia, or Nocardia. The pathogenic organism can be any one or more of the organisms listed in Table 3. The one or more symptoms can comprise delusional thought, seizures, speech disorders, movement difficulty, or aggression.

Disclosed herein is a method for identifying a subject as having a risk of autoantibody associated disease comprising: obtaining a biological sample from said subject; assaying said biological sample for antibodies that bind to the amino acid sequence LQNRKLV (SEQ ID NO: 41); and optionally, outputting a report or identifying said subject as being at high risk or low risk for said autoantibody associated disease. The biological sample can comprise blood, sweat, saliva, cerebrospinal fluid (CSF), amniotic fluid, or mucus. The subject can be pregnant. The risk of autoantibody associated disease can be associated with a fetus of said subject. The method can further comprise treating said subject for said autoantibody associated disease. The treating can comprise administering an FcRn receptor blocking compound to said subject. The FcRn receptor blocking compound can be a polypeptide. The FcRn receptor blocking compound can be an antibody or antigen binding fragment thereof. The FcRn receptor blocking compound can be selected from the group consisting of: Rozanolixizumab, SYNT001, M281, Argx-113, HL161-11G, HL161-11H, HL161-1 A, DX-2504, DX-2507, ABY039, IMVT-1401/RVT1401, and combinations thereof.

Disclosed herein is a method for the treatment or improvement of a psychiatric or central nervous system disease or disorder comprising administering to a subject in need thereof an antibody or fragment that binds to a N-methyl-D-aspartate (NMDA) receptor of said subject. The antibody or antigen binding fragment thereof can bind a NMDAR1 (NR1) subunit of the NMDA receptor. The antibody or antigen binding fragment thereof can bind an epitope of NR1 comprising amino acid sequence LQNRKLV (SEQ ID NO: 41). The antibody or antigen binding fragment thereof can bind at least one amino acid of NR1 consisting of amino acid sequence LQNRKLV (SEQ ID NO: 41).

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates the binding of a pathogenic autoantibody fragment as described herein to the NR1 subunit of a NMDA receptor.

FIG. 2 illustrates the residues of an antibody fragment as described herein interacting with the amino acid residues of the NR1 subunit of an NMDA receptor.

FIG. 3 illustrates a hydrogen deuterium exchange analysis of the binding of an antibody as described herein to the N-terminal of an NR1 subunit of an NMDA receptor . . .

FIG. 4 illustrates the predicted epitope structure of an NR1 subunit of an NMDA receptor.

FIG. 5 illustrates the HDX mass spectrometry analysis of the anti-NR1 subunit and a therapeutic antibody.

FIG. 6 illustrates the HDX mass spectrometry analysis of the anti-NR1 subunit and a pathogenic autoantibody.

FIG. 7 illustrates the whole internal kinesin motor domain protein sequence of Toxoplasma gondii.

FIGS. 8A and 8B illustrate a surface probability plot and associated antigenic index over the core epitope peptide sequence of the NMDA receptor NR1 subunit (8A) as compared to the Toxoplasma gondii kinesin protein (8B).

### DETAILED DESCRIPTION

In the following description, certain specific details are set forth in order to provide a thorough understanding of various embodiments. However, one skilled in the art will understand that the embodiments provided may be practiced without these details. Unless the context requires otherwise, throughout the specification and claims which follow, the word “comprise” and variations thereof, such as, “comprises” and “comprising” are to be construed in an open, inclusive sense, that is, as “including, but not limited to.” As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. Further, headings provided herein are for convenience only and do not interpret the scope or meaning of the claimed embodiments.

Although certain embodiments and examples are disclosed below, inventive subject matter extends beyond the specifically disclosed embodiments to other alternative embodiments and/or uses, and to modifications and equivalents thereof. Thus, the scope of the claims appended hereto is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments, however, the order of description should not be construed to imply that these operations are order dependent. Additionally, the structures, systems, and/or devices described herein may be embodied as integrated components or as separate components.

For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

As used herein in the specification and claims, including as used in the examples and unless otherwise expressly specified, all numbers may be read as if prefaced by the word “about” or “approximately,” even if the term does not expressly appear. As used herein the term “about” refers to an amount that is near the stated amount by 10% or less. In the following detailed description, reference is made to the accompanying figures, which form a part hereof. In the figures, similar symbols typically identify similar components, unless context dictates otherwise. The illustrative embodiments described in the detailed description, figures, and claims are not meant to be limiting. Other embodiments may be utilized, and other changes may be made, without departing from the scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein the term “individual,” “patient,” or “subject” refers to individuals diagnosed with, suspected of being afflicted with, or at-risk of developing at least one disease for which the described compositions and method are useful for treating. In certain embodiments the individual is a mammal. In certain embodiments, the mammal is a mouse, rat, rabbit, dog, cat, horse, cow, sheep, pig, goat, llama, alpaca, or yak. In certain embodiments, the individual is a human.

Among the provided antibodies are monoclonal antibodies, and antibody fragments. The antibodies include antibody-conjugates and molecules comprising the antibodies, such as chimeric molecules. Thus, an antibody includes, but is not limited to, full-length and native antibodies, as well as fragments and portion thereof retaining the binding specificities thereof, such as any specific binding portion thereof including those having any number of, immunoglobulin classes and/or isotypes (e.g., IgG1, IgG2, IgG3, IgG4, IgM, IgA, IgD, IgE and IgM); and biologically relevant (antigen-binding) fragments or specific binding portions thereof, including but not limited to Fab, F(ab′)₂, Fv, and scFv (single chain or related entity). A monoclonal antibody is generally one within a composition of substantially homogeneous antibodies; thus, any individual antibodies comprised within the monoclonal antibody composition are identical except for possible naturally occurring mutations that may be present in minor amounts. The monoclonal antibody can comprise a human IgG1 constant region. The monoclonal antibody can comprise a human IgG4 constant region.

The term “antibody” herein is used in the broadest sense and includes monoclonal antibodies, and includes intact antibodies and functional (antigen-binding) antibody fragments thereof, including fragment antigen binding (Fab) fragments, F(ab′)₂ fragments, Fab′ fragments, Fv fragments, recombinant IgG (rlgG) fragments, single chain antibody fragments, including single chain variable fragments (sFv or scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD. The antibody can comprise a human IgG1 constant region. The antibody can comprise a human IgG4 constant region.

The terms “complementarity determining region,” and “CDR,” which are synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4). The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273,927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc MP et al., “IMGT unique numbering for immunoglobulin and T cell receptor variable domains and Ig superfamily V-like domains,” Dev Comp Immunol, 2003 January;27 (1): 55-77 (“IMGT” numbering scheme); Honegger A and Plückthun A, “Yet another numbering scheme for immunoglobulin variable domains: an automatic modeling and analysis tool,” J Mol Biol, 2001 Jun. 8;309 (3): 657-70, (“Aho” numbering scheme); and Whitelegg NR and Rees AR, “WAM: an improved algorithm for modelling antibodies on the WEB,” Protein Eng. 2000 December; 13 (12): 819-24 (“AbM” numbering scheme. In certain embodiments, the CDRs of the antibodies described herein can be defined by a method selected from Kabat, Chothia, IMGT, Aho, AbM, or combinations thereof.

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme.

The term “variable region” or “variable domain” refers to the domain of an antibody heavy or light chain that is involved in binding the antibody to antigen. The variable domains of the heavy chain and light chain (V_{H} and V_{L}, respectively) of a native antibody generally have similar structures, with each domain comprising four conserved framework regions (FRs) and three CDRs (See e.g., Kindt et al. Kuby Immunology, 6th ed., W. H. Freeman and Co., page 91 (2007)). A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively (See e.g., Portolano et al., J. Immunol. 150:880-887 (1993); Clarkson et al., Nature 352:624-628 (1991)).

Specific binding or binding of antibody molecules described herein refers to binding mediated by one or more CDR portions of the antibody. Not all CDRs may be required for specific binding. Specific binding can be demonstrated for example by an ELISA against a specific recited target or antigen that shows significant increase in binding compared to an isotype control antibody.

As described herein an “epitope” refers to the binding determinant of an antibody or fragment described herein minimally necessary for specific binding of the antibody or antigen binding fragment thereof to a target antigen. When the target antigen is a polypeptide, the epitope will be a continuous or discontinuous epitope. A continuous epitope is formed by one region of the target antigen, while a discontinuous epitope may be formed from two or more separate regions. A discontinuous epitope, for example, may form when a target antigen adopts a tertiary structure that brings two amino acid sequences together and forms a three-dimensional structure bound by the antibody. When the target antigen is a polypeptide, the epitope will generally be a plurality of amino acids linked into a polypeptide chain. A continuous epitope may comprise 5, 6, 7, 8, 9, 10, 11, 12, 13, 14, 15, 16, 17, 18, 19, or 20 contiguous amino acids. While an epitope may comprise a contiguous polymer of amino acids, not every amino acid of the polymer may be contacted by an amino acid residue of the antibody. Such non-contacted amino acids will still comprise part of the epitope as they may be important for the structure and linkage of the contacted amino acids. The skilled artisan may determine if any given antibody binds an epitope of a reference antibody, for example, by cross-blocking experiments with a reference antibody. In certain embodiments, described herein, are antibodies that bind the same epitope of the described antibodies. In certain embodiments, described herein, are antibodies that are competitively blocked by the described antibodies. In certain embodiments, described herein, are antibodies that compete for binding with the described antibodies.

Among the provided antibodies are antibody fragments. An “antibody fragment” refers to a molecule other than an intact antibody that comprises a portion of an intact antibody that binds the antigen to which the intact antibody binds. Examples of antibody fragments include, but are not limited to, Fv, Fab, Fab′, Fab′-SH, F(ab′)₂; diabodies; linear antibodies; single-chain antibody molecules (e.g., scFv or sFv); and multispecific antibodies formed from antibody fragments. In particular embodiments, the antibodies are single-chain antibody fragments comprising a variable heavy chain region and/or a variable light chain region, such as scFvs.

Antibody fragments can be made by various techniques, including but not limited to proteolytic digestion of an intact antibody as well as production by recombinant host cells. In some embodiments, the antibodies are recombinantly-produced fragments, such as fragments comprising arrangements that do not occur naturally, such as those with two or more antibody regions or chains joined by synthetic linkers, e.g., polypeptide linkers, and/or those that are not produced by enzyme digestion of a naturally occurring intact antibody. In some aspects, the antibody fragments are scFvs.

A “humanized” antibody is an antibody in which all or substantially all CDR amino acid residues are derived from non-human CDRs and all or substantially all FR amino acid residues are derived from human FRs. A humanized antibody optionally may include at least a portion of an antibody constant region derived from a human antibody. A “humanized form” of a non-human antibody refers to a variant of the non-human antibody that has undergone humanization, typically to reduce immunogenicity to humans, while retaining the specificity and affinity of the parental non-human antibody. In some embodiments, some FR residues in a humanized antibody are substituted with corresponding residues from a non-human antibody (e.g., the antibody from which the CDR residues are derived), e.g., to restore or improve antibody specificity or affinity.

Among the provided antibodies are human antibodies. A “human antibody” is an antibody with an amino acid sequence corresponding to that of an antibody produced by a human or a human cell, or non-human source that utilizes human antibody repertoires or other human antibody-encoding sequences, including human antibody libraries. The term excludes humanized forms of non-human antibodies comprising non-human antigen-binding regions, such as those in which all or substantially all CDRs are non-human.

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic animals, the endogenous immunoglobulin loci have generally been inactivated. Human antibodies also may be derived from human antibody libraries, including phage display and cell-free libraries, containing antibody-encoding sequences derived from a human repertoire.

The terms “polypeptide” and “protein” are used interchangeably to refer to a polymer of amino acid residues, and are not limited to a minimum length. Polypeptides, including the provided antibodies and antibody chains and other peptides, e.g., linkers and binding peptides, may include amino acid residues including natural and/or non-natural amino acid residues. The terms also include post-expression modifications of the polypeptide, for example, glycosylation, sialylation, acetylation, phosphorylation, and the like. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification. In some embodiments, amino acid sequence variants of the antibodies provided herein are contemplated. A variant typically differs from a polypeptide specifically disclosed herein in one or more substitutions, deletions, additions and/or insertions. Such variants can be naturally occurring or can be synthetically generated, for example, by modifying one or more of the above polypeptide sequences of the invention and evaluating one or more biological activities of the polypeptide as described herein and/or using any of a number of known techniques. For example, it may be desirable to improve the binding affinity and/or other biological properties of the antibody amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

Percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known for instance, using publicly available computer software such as BLAST, BLAST-2, ALIGN or Megalign (DNASTAR) software. Appropriate parameters for aligning sequences are able to be determined, including algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

In some embodiments, methods described herein comprise the treatment or improvement of a psychiatric or central nervous system disease or disorder. In some embodiments, the psychiatric or central nervous system disease or disorder is an autoantibody associated disorder. In some embodiments, the psychiatric or central nervous system disease or disorder is a neurodegenerative or cognitive disease or disorder. In some embodiments, the psychiatric or central nervous system disease or disorder can be schizophrenia, psychosis, bipolar disorder, depression, epilepsy, or dementia.

In some embodiments, methods described herein comprise administering to a subject in need thereof an antibody or antigen binding fragment thereof binds to a neuron or glia. In some embodiments, antibody or antigen binding fragment thereof binds to a neuronal or glial receptor. In some embodiments, the neuronal or glial receptor is a ligand-gated cation channel. In some embodiments, the ligand-gated cation channel is activated by glutamate. In some embodiments, the neuronal receptor is a G-protein coupled ionotropic glutamate receptor. In some embodiments, the antibody or antigen binding fragment thereof binds to a NMDAR1 (NR1) subunit of the NMDA receptor. In some embodiments, the antibody or antigen binding fragment thereof binds to at least one amino acid of an epitope of NR1 consisting of amino acid sequence LQNRKLV (SEQ ID NO: 41). In some embodiments, the antibody or antigen binding fragment thereof binds to at least two amino acids of an epitope of NR1 consisting of amino acid sequence LQNRKLV (SEQ ID NO: 41).

In some embodiments, the antibody or antigen binding fragment thereof binds to at least three amino acids of an epitope of NR1 consisting of amino acid sequence LQNRKLV (SEQ ID NO: 41).

In some embodiments, the antibody or antigen binding fragment thereof comprises a heavy chain with at least 80%, 85%, 90%, 95%, or 98% identity to SEQ ID NO: 1, 3, 5, 7, or 9. In some embodiments, the antibody or antigen binding fragment thereof comprises a heavy chain with CDR regions with at least 80%, 85%, 90%, 95%, or 98% identity to SEQ ID NO: 11, 12, 13, 17, 18, 19, 23, 24, 25, 29, 30, 31, 35, 36, or 37.

In some embodiments, the antibody or antigen binding fragment thereof comprises a light chain with at least 80%, 85%, 90%, 95%, or 98% identity to SEQ ID NO: 2, 4, 6, 8, or 10. In some embodiments, the antibody or fragment thereof comprises a light chain with CDR regions with at least 80%, 85%, 90%, 95%, or 98% identity to SEQ ID NO: 14, 15, 16, 20, 21, 22, 26, 27, 28, 32, 33, 34, 38, 39, or 40.

In some embodiments, the antibody or antigen binding fragment thereof comprises a heavy chain variable region and a light chain variable region, the heavy and light chain variable region comprising: a heavy chain CDR1 as set forth in SEQ ID NO: 11, a heavy chain CDR2 as set forth in SEQ ID NO: 12, a heavy chain CDR3 as set forth in SEQ ID NO: 13, a light chain CDR1 as set forth in SEQ ID NO: 14, a light chain CDR2 as set forth in SEQ ID NO:15, and/or a light chain CDR3 as set forth in SEQ ID NO:16.

In some embodiments, the antibody or antigen binding fragment thereof comprises a heavy chain variable region and a light chain variable region, the heavy and light chain variable region comprising: a heavy chain CDR1 as set forth in SEQ ID NO: 17, a heavy chain CDR2 as set forth in SEQ ID NO: 18, a heavy chain CDR3 as set forth in SEQ ID NO: 19, a light chain CDR1 as set forth in SEQ ID NO: 20, a light chain CDR2 as set forth in SEQ ID NO:21, and/or a light chain CDR3 as set forth in SEQ ID NO:22.

In some embodiments, the antibody or antigen binding fragment thereof comprises a heavy chain variable region and a light chain variable region, the heavy and light chain variable region comprising: a heavy chain CDR1 as set forth in SEQ ID NO:23, a heavy chain CDR2 as set forth in SEQ ID NO: 24, a heavy chain CDR3 as set forth in SEQ ID NO: 25, a light chain CDR1 as set forth in SEQ ID NO: 26, a light chain CDR2 as set forth in SEQ ID NO:27, and/or a light chain CDR3 as set forth in SEQ ID NO:28.

In some embodiments, the antibody or antigen binding fragment thereof comprises a heavy chain variable region and a light chain variable region, the heavy and light chain variable region comprising: a heavy chain CDR1 as set forth in SEQ ID NO: 29, a heavy chain CDR2 as set forth in SEQ ID NO: 30, a heavy chain CDR3 as set forth in SEQ ID NO: 31, a light chain CDR1 as set forth in SEQ ID NO: 32, a light chain CDR2 as set forth in SEQ ID NO:33, and/or a light chain CDR3 as set forth in SEQ ID NO:34.

In some embodiments, the antibody or antigen binding fragment thereof comprises a heavy chain variable region and a light chain variable region, the heavy and light chain variable region comprising: a heavy chain CDR1 as set forth in SEQ ID NO: 35, a heavy chain CDR2 as set forth in SEQ ID NO: 36, a heavy chain CDR3 as set forth in SEQ ID NO: 37, a light chain CDR1 as set forth in SEQ ID NO: 38, a light chain CDR2 as set forth in SEQ ID NO:39, and/or a light chain CDR3 as set forth in SEQ ID NO:40.

In some embodiments, the antibody or antigen binding fragment thereof comprises a heavy chain with at least 80%, 85%, 90%, 95%, or 98%, 99%, or 100% identity to SEQ ID NO: 1, 3, 5, 7, or 9, and a light chain with at least 80%, 85%, 90%, 95%, or 98%, 99%, or 100% identity to SEQ ID NO: 2, 4, 6, 8, or 10.

In some embodiments, the antibody or antigen binding fragment thereof comprises a heavy chain with at least 80%, 85%, 90%, 95%, or 98%, 99%, or 100% identity to SEQ ID NO: land a light chain with at least 80%, 85%, 90%, 95%, or 98%, 99%, or 100% identity to SEQ ID NO: 2.

In some embodiments, the antibody or antigen binding fragment thereof comprises a heavy chain with at least 80%, 85%, 90%, 95%, or 98%, 99%, or 100% identity to SEQ ID NO: 3 and a light chain with at least 80%, 85%, 90%, 95%, or 98%, 99%, or 100% identity to SEQ ID NO: 4.

In some embodiments, the antibody or antigen binding fragment thereof comprises a heavy chain with at least 80%, 85%, 90%, 95%, or 98%, 99%, or 100% identity to SEQ ID NO: 5, and a light chain with at least 80%, 85%, 90%, 95%, or 98%, 99%, or 100% identity to SEQ ID NO:

6.

In some embodiments, the antibody or antigen binding fragment thereof comprises a heavy chain with at least 80%, 85%, 90%, 95%, or 98%, 99%, or 100% identity to SEQ ID NO: 7, or 9, and a light chain with at least 80%, 85%, 90%, 95%, or 98%, 99%, or 100% identity to SEQ ID NO: 8.

In some embodiments, the antibody or antigen binding fragment thereof comprises a heavy chain with at least 80%, 85%, 90%, 95%, or 98%, 99%, or 100% identity to SEQ ID NO: 9, and a light chain with at least 80%, 85%, 90%, 95%, or 98%, 99%, or 100% identity to SEQ ID NO: 10.

TABLE 1
Anti-NR1 antibody sequences

| | | SEQ | AA Sequence (CDRs | SEQ | |
|---|---|---|---|---|---|
| Antibody | Region | ID No. | underlined) | ID No. | Nucleotide Sequence |
| α hu | HC | 1 | QVQLQ ESGPG | 42 | caggtccagctgcaagagtctggccctg |
| NR1 | | | LVKPS GTLSL | | gactggtcaagccttctggcaccctgtctc |
| clone 1 | | | TCAVS GGSIS | | tgacatgtgctgtgtccggcggctccatct |
| | | | SSNWW SWVRQ | | cctcctctaattggtggtcttgggtccgaca |
| | | | PPGKG LEWIG | | gcctcctggcaaaggactggaatggatc |
| | | | EIYHS GNTNY | | ggcgagatctaccactccggcaacacca |
| | | | NPSLK SRVTV | | actacaaccccagcctgaagtccagagtg |
| | | | SVDKS KNQFS | | accgtgtccgtggacaagtccaagaacc |
| | | | LKLTS VTAAD | | agttctccctgaagctgacctctgtgaccg |
| | | | TAVYY CARDV | | ctgccgataccgccgtgtactactgtgcta |
| | | | SGGVN WFDPW | | gagatgtgtctggcggagtgaattggttcg |
| | | | GQGTL VTVSS | | atccttggggccagggcacactggttacc |
| | | | | | gtgtcctct |
| α hu | LC | 2 | NFMLT QPHSV | 43 | aactttatgctgacccagcctcactccgtgt |
| NR1 | | | SESPG KTVTI | | ccgagtctccaggcaagaccgtgaccat |
| clone 1 | | | SCTRS SGSIA | | ctcctgtaccagatcctccggctctatcgc |
| | | | SNYVQ WYQQR | | ctccaactacgtgcagtggtatcagcaga |
| | | | PGSAP TTVIY | | ggcctggctctgctcctaccaccgtgatct |
| | | | EDNQR PSGVP | | acgaggacaaccagaggccttctggcgt |
| | | | DRFSG SIDSS | | gcccgataggttctctggctccatcgactc |
| | | | SNSAS LTISG | | ctcttccaactccgcctctctgaccatcagc |
| | | | LKTED EADYY | | ggcctgaaaaccgaggacgaggccgac |
| | | | CQSYD SSTVV | | tactactgccagtcctacgactcttccacc |
| | | | FGGGT KLTVL | | gtggtgtttggcggcggaacaaagctgac |
| | | | | | agtgctg |
| α hu | HC | 3 | EVQLV QSGAE | 44 | gaagttcagctggttcagtctggcgccga |
| NR1 | | | VKKPG ESLKI | | agtgaagaagcctggcgagtccctgaag |
| clone 2 | | | SCKGS GYTYT | | atctcctgcaaaggctccggctacaccta |
| | | | SYWMN WVRQM | | caccagctactggatgaactgggtccgac |
| | | | PGKGL EWMGR | | agatgcctggcaaaggcctggaatggat |
| | | | IDPYD SETHY | | gggcagaatcgacccctacgactccgag |
| | | | DQKFK DQVTL | | acacactacgaccagaaattcaaggacca |
| | | | SVDKS ISTAY | | agtgaccctgagcgtggacaagtccatct |
| | | | LQWSS LKASD | | ccaccgcttacctgcagtggtcctctctga |
| | | | TAMYY CAGGI | | aggcctctgacaccgccatgtactactgtg |
| | | | TTILG GYFDY | | ctggcggcatcaccaccatcctcggcgg |
| | | | WGQGT LVTVS S | | ctactttgattactggggccagggcacact |
| | | | | | ggtcaccgtttcttcc |
| α hu | LC | 4 | DIQMT QSPSS | 45 | gacatccagatgacccagtctccatcctct |
| NR1 | | | LSASV GDRVT | | ctgtccgcctctgtgggcgacagagtgac |
| clone 2 | | | ITCRA SQDIS | | catcacctgtagagccagccaggacatct |
| | | | NYLNW YQQKP | | ccaactacctgaactggtatcagcagaag |
| | | | GKAPK LLIYY | | cccggcaaggcccctaagctgctgatcta |
| | | | TSRLH SGVPS | | ctacacctctcggctgcactctggcgtgcc |
| | | | RFSGS GSGTD | | ctctagattttctggctccggctctggcacc |
| | | | YTLTI SSLQP | | gactataccctgacaatctccagcctgca |
| | | | EDFAT YYCQQ | | gcctgaggacttcgccacctactattgcca |
| | | | GNTLP PYTFG | | gcagggcaacaccctgcctccatacacat |
| | | | QGTKV EIKR | | ttggccagggcaccaaggtggaaatcaa |
| | | | | | gcgt |
| α hu | HC | 5 | EVQLV ESGGG | 46 | gaagtgcagctggttgaatctggcggcg |
| NR1 | | | LVQPG GSLRL | | gattggttcagcctggcggatctctgagac |
| clone 3 | | | SCKAS GYAYT | | tgtcctgcaaggcttctggctacgcctaca |
| | | | SYWMN WVRQA | | cctcctactggatgaactgggtccgacag |
| | | | PGKGL VWVGR | | gctcctggcaaaggactcgtgtgggtcgg |
| | | | IDPYD SETHY | | aagaatcgacccctacgactccgagaca |
| | | | NQKFK DRFTL | | cactacaaccagaagttcaaggaccggtt |
| | | | SVDKA KSTTY | | caccctgagcgtggacaaggccaagtct |
| | | | LQMNS LRAED | | accacctacctgcagatgaactccctgag |
| | | | TAVYY CATLI | | agccgaggacaccgccgtgtactattgcg |
| | | | TTLRG EGAME | | ctaccctgatcaccacactgagaggcga |
| | | | YWGQG TLVTV SS | | gggcgccatggaatattggggacaggga |
| | | | | | accctggtcaccgtgtcctct |
| α hu | LC | 6 | DLQMT QSPSS | 47 | gacctgcagatgacccagtctccttccag |
| NR1 | | | LSASV GDRVT | | cctgtctgcctctgtgggcgacagagtga |
| clone 3 | | | ITCKA SQDVN | | ccatcacatgcaaggccagccaggacgt |
| | | | TAVAW YQQKP | | gaacaccgccgttgcttggtatcagcaga |
| | | | GKAPK LLIYW | | agcctggcaaggcccctaagctgctgatc |
| | | | ASTRH TGVPS | | tactgggcctccaccagacataccggcgt |
| | | | RFSGS GSGTD | | gccctctagattctccggctctggctctgg |
| | | | YTLTI SSLQP | | caccgactataccctgacaatctccagcct |
| | | | EDFAT YYCQQ | | gcagcctgaggacttcgccacctactact |
| | | | HYSTP FTFGQ | | gccagcagcactacagcacccctttcacc |
| | | | GTKVE IKR | | tttggccagggcaccaaggtggaaatcaa |
| | | | | | gcgt |
| α hu | HC | 7 | QVQLQ ESGPG | 48 | caggttcagctgcaagagtctggccctgg |
| NR1 | | | LVKPS ETLSL | | cctggtcaagccttccgaaacactgtccct |
| clone 4 | | | TCSVT GYSIT | | gacctgcagcgtgaccggctactctatca |
| | | | SDYYW HWIRQ | | cctccgactactactggcactggatcaga |
| | | | PPGKG LEWIA | | cagcctccaggcaaaggcctggaatgga |
| | | | YIRYD GRNDY | | tcgcctacatcagatacgacggccggaa |
| | | | NPSLK NRVTI | | cgactacaaccccagcctgaagaacaga |
| | | | SRDTS KNQFS | | gtgaccatcagccgggacacctccaaga |
| | | | LKLSS VTAAD | | accagttctccctgaagctgtcctccgtga |
| | | | TAVYY CARED | | ccgctgctgataccgccgtgtactactgc |
| | | | YGSSS FDYWG | | gccagagaggactacggctcctcctccttt |
| | | | QGTLV TVSS | | gattactggggccagggcaccctggtca |
| | | | | | ccgttagttct |
| α hu | LC | 8 | DIQMT QSPSS | 49 | gacatccagatgacccagtctccatcctct |
| NR1 | | | LSASV GDRVT | | ctgtccgcctctgtgggcgacagagtgac |
| clone 4 | | | ITCRA SENIY | | catcacctgtcgggcctccgagaacatct |
| | | | SNLAW YQQKP | | actccaacctggcctggtatcagcagaag |
| | | | GKAPK LLVYA | | cctggcaaggctcctaagctgctggtgta |
| | | | ATNLA DGVPS | | cgccgctaccaatctggctgatggcgtgc |
| | | | RFSGS GSGTD | | cctctagattctccggctctggctctggca |
| | | | YTLTI SSLQP | | ccgactataccctgacaatctccagcctgc |
| | | | EDFAT YYCQH | | agcctgaggacttcgccacctactactgc |
| | | | FWDSP PTFGQ | | cagcacttctgggacagccctccaaccttt |
| | | | GTKVE IKR | | ggccagggcaccaaggtggaaatcaag |
| | | | | | cgt |
| α hu | HC | 9 | QVQLQ ESGPG | 50 | caggttcagctgcaagagtctggccctgg |
| NR1 | | | LVKPS QTLSL | | cctggtcaagccttctcagaccctgtctct |
| clone 5 | | | TCSVT GYSIT | | gacctgcagcgtgaccggctactccatca |
| | | | SDYYW HWIRQ | | cctccgactactactggcactggatcaga |
| | | | PPGKG LEWIA | | cagcctccaggcaaaggcctggaatgga |
| | | | YIRYD GRNDS | | tcgcctacatcagatacgacggccggaa |
| | | | NPSLK NRVTI | | cgacagcaaccccagcctgaagaacaga |
| | | | SRDTS KNQFS | | gtgaccatcagccgggacacctccaaga |
| | | | LKLSS VTAAD | | accagttctccctgaagctgtcctccgtga |
| | | | TAVYY CARED | | ccgctgctgataccgccgtgtactactgc |
| | | | YASSS FDYWG | | gccagagaggactacgcctcctcctccttt |
| | | | QGTLV TVSS | | gattactggggccagggcaccctggtca |
| | | | | | ccgttagttct |
| α hu | LC | 10 | DIQMT QSPSS | 51 | gacatccagatgacccagtctccatcctct |
| NR1 | | | LSASV GDRVT | | ctgtccgcctctgtgggcgacagagtgac |
| clone 5 | | | ITCRP SENIY | | catcacctgtcggccttccgagaacatcta |
| | | | SNLAW YQQKP | | ctccaacctggcctggtatcagcagaagc |
| | | | GKAPK LLVYA | | ctggcaaggctcctaagctgctggtgtac |
| | | | ATNLA DGVPS | | gccgctaccaatctggctgatggcgtgcc |
| | | | RFSGS GSGTD | | ctctagattctccggctctggctctggcac |
| | | | YTLTI SSLQP | | cgactataccctgacaatctccagcctgca |
| | | | EDIAT YYCQH | | gcctgaggatatcgccacctactactgcc |
| | | | FWDSP PTFGQ | | agcacttctgggacagccctccaacctttg |
| | | | GTKLE IKR | | gccagggcaccaagctggaaatcaagc |
| | | | | | gt |
| α hu | HC | 11 | GGSISSSNW | | |
| NR1 | CDR1 | | | | |
| α hu | HC | 12 | YHSGNTN | | |
| NR1 | CDR2 | | | | |
| α hu | HC | 13 | ARDVSGGVNWFD | | |
| NR1 | CDR3 | | P | | |
| α hu | LC | 14 | SGSIASNY | | |
| NR1 | CDR1 | | | | |
| α hu | LC | 15 | EDN | | |
| NR1 | CDR2 | | | | |
| α hu | LC | 16 | QSYDSSTVV | | |
| NR1 | CDR3 | | | | |
| α hu | HC | 17 | SYWMN | | |
| NR1 | CDR1 | | | | |
| α hu | HC | 18 | RIDPYDSETHYDQ | | |
| NR1 | CDR2 | | KFKD | | |
| α hu | HC | 19 | GITTILGGYFDY | | |
| NR1 | CDR3 | | | | |
| α hu | LC | 20 | RASQDISNYLN | | |
| NR1 | CDR1 | | | | |
| α hu | LC | 21 | YTSRLHS | | |
| NR1 | CDR2 | | | | |
| α hu | LC | 22 | QQGNTLPPYT | | |
| NR1 | CDR3 | | | | |
| α hu | HC | 23 | SYWMN | | |
| NR1 | CDR1 | | | | |
| α hu | HC | 24 | RIDPYDSETHYNQ | | |
| NR1 | CDR2 | | KFKD | | |
| α hu | HC | 25 | LITTLRGEGAMEY | | |
| NR1 | CDR3 | | | | |
| α hu | LC | 26 | KASQDVNTAVA | | |
| NR1 | CDR1 | | | | |
| α hu | LC | 27 | WASTRHT | | |
| NR1 | CDR2 | | | | |
| α hu | LC | 28 | QQHYSTPFT | | |
| NR1 | CDR3 | | | | |
| α hu | HC | 29 | SDYYWH | | |
| NR1 | CDR1 | | | | |
| α hu | HC | 30 | YIRYDGRNDYNPS | | |
| NR1 | CDR2 | | LKNRVTIS | | |
| α hu | HC | 31 | SSFDYWGQGT | | |
| NR1 | CDR3 | | | | |
| α hu | LC | 32 | RASENIYSNLA | | |
| NR1 | CDR1 | | | | |
| α hu | LC | 33 | AATNLAD | | |
| NR1 | CDR2 | | | | |
| α hu | LC | 34 | QHFWDSPPT | | |
| NR1 | CDR3 | | | | |
| α hu | HC | 35 | SDYYWH | | |
| NR1 | CDR1 | | | | |
| α hu | HC | 36 | YIRYDGRNDSNPS | | |
| NR1 | CDR2 | | LKN | | |
| α hu | HC | 37 | EDYASSSFDY | | |
| NR1 | CDR3 | | | | |
| α hu | LC | 38 | RPSENIYSNLA | | |
| NR1 | CDR1 | | | | |
| α hu | LC | 39 | AATNLAD | | |
| NR1 | CDR2 | | | | |
| α hu | LC | 40 | QHFWDSPPT | | |
| NR1 | CDR3 | | | | |

In some embodiments, methods described herein comprise identifying an antibody or fragment thereof for use in treatment of a psychiatric or central nervous system disease or disorder. Antibodies or fragments thereof can be identified by assaying for affinity to an antigenic peptide of a pathogenic organism. Antibodies or fragments thereof can be identified by assaying for affinity to an epitope LQNRKLV (SEQ ID NO: 41) of a NR1 subunit of a NMDA receptor.

In some embodiments, pathogenic organisms can include the genus of Toxoplasma, Paramecium, Campylobacter, Enterococcus, Peptoniphilus, Paenalcaligenes, Pseudomonas, Burkholderia, Chromobacterium, Acinetobacter, Paenibacillus, Escherichia, or Nocardia. In some embodiments, the pathogenic organism can be any one or more of the organisms listed in Table 3.

In some embodiments, methods described herein comprise identifying a subject as having been exposed to a pathogenic organism. Identifying can comprise assaying a biological sample obtained from the subject for an antibody that binds to an immunogenic epitope of a pathogenic organism. The method can comprise identifying said subject as having one or more symptoms of a psychiatric or central nervous system disease or disorder. The method can further comprise outputting a report identifying said subject as being at high risk for N-methyl-D-aspartate (NMDA) receptor dysfunction.

In some embodiments, the one or more symptoms of psychiatric or central nervous system disease or disorder can comprise delusional thought, seizures, speech disorders, movement difficulty, or aggression.

In some embodiments, the biological sample can be a fluid derived from the subject. The biological sample can comprise blood, sweat, saliva, cerebrospinal fluid (CSF), amniotic fluid, or mucus. In some embodiments, the biological sample comprises blood, plasma, or serum. In some embodiments, the biological sample comprises plasma. In some embodiments, the biological sample comprises serum.

In some embodiments, methods described herein can comprise identifying a subject as having a risk of autoantibody associated disease comprising: obtaining a biological sample from said subject; assaying said biological sample for antibodies that bind to the amino acid sequence LQNRKLV (SEQ ID NO: 41); and outputting a report identifying said subject as being at high risk or low risk for said autoantibody associated disease.

In some embodiments, methods disclosed herein comprise administering to a subject in need thereof an antibody or fragment that binds to a N-methyl-D-aspartate (NMDA) receptor of the subject.

In some embodiments, the subject is pregnant. In some embodiments, a risk of autoantibody associated disease can be associated with a fetus of the pregnant subject. In some embodiments, the methods comprise treating the pregnant subject for the autoantibody associated disease. In some embodiments, treating can comprise administering an FcRn receptor blocking compound to said subject.

In some embodiments, the FcRn receptor blocking compound can be a polypeptide. In some embodiments, the FcRn receptor blocking compound can be an antibody or fragment thereof. In some embodiments, the FcRn receptor blocking compound can be selected from the group consisting of: Rozanolixizumab, SYNT001, M281, Argx-113, HL161-11G, HL161-11H, HL 161-1 A, DX-2504, DX-2507, ABY039, IMVT-1401/RVT1401, and combinations thereof.

Alterations (e.g., substitutions) may be made in CDRs, e.g., to improve antibody affinity. Such alterations may be made in CDR encoding codons with a high mutation rate during somatic maturation (See e.g., Chowdhury, Methods Mol. Biol. 207:179-196 (2008)), and the resulting variant can be tested for binding affinity. Affinity maturation (e.g., using error-prone PCR, chain shuffling, randomization of CDRs, or oligonucleotide-directed mutagenesis) can be used to improve antibody affinity (See e.g., Hoogenboom et al. in Methods in Molecular Biology 178:1-37 (2001)). CDR residues involved in antigen binding may be specifically identified, e.g., using alanine scanning mutagenesis or modeling (See e.g., Cunningham and Wells Science, 244:1081-1085 (1989)). CDR-H3 and CDR-L3 in particular are often targeted. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions and deletions include amino- and/or carboxyl-terminal fusions ranging in length from one residue to polypeptides containing a hundred or more residues, as well as intrasequence insertions and deletions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody. Examples of intrasequence insertion variants of the antibody molecules include an insertion of 3 amino acids in the light chain. Examples of terminal deletions include an antibody with a deletion of 7 or less amino acids at an end of the light chain.

In some embodiments, the antibodies are altered to increase or decrease their glycosylation (e.g., by altering the amino acid sequence such that one or more glycosylation sites are created or removed). A carbohydrate attached to an Fc region of an antibody may be altered. Native antibodies from mammalian cells typically comprise a branched, biantennary oligosaccharide attached by an N-linkage to Asn297 of the CH2 domain of the Fc region (See e.g., Wright et al. TIBTECH 15:26-32 (1997)). The oligosaccharide can be various carbohydrates, e.g., mannose, N-acetyl glucosamine (GlcNAc), galactose, sialic acid, fucose attached to a GlcNAc in the stem of the biantennar oligosaccharide structure. Modifications of the oligosaccharide in an antibody can be made, for example, to create antibody variants with certain improved properties. Antibody glycosylation variants can have improved ADCC and/or CDC function. In some embodiments, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn297 (See e.g., WO 08/077546). Asn297 refers to the asparagine residue located at about position 297 in the Fc region (EU numbering of Fc region residues; See e.g., Edelman et al. Proc Natl Acad Sci USA. 1969 May; 63 (1): 78-85). However, Asn297 may also be located about +3 amino acids upstream or downstream of position 297, i.e., between positions 294 and 300, due to minor sequence variations in antibodies. Such fucosylation variants can have improved ADCC function (See e.g., Okazaki et al. J. Mol. Biol. 336:1239-1249 (2004); and Yamane-Ohnuki et al. Biotech. Bioeng. 87:614 (2004)). Cell lines, e.g., knockout cell lines and methods of their use can be used to produce defucosylated antibodies, e.g., Lec13 CHO cells deficient in protein fucosylation and alpha-1,6-fucosyltransferase gene (FUT8) knockout CHO cells (See e.g., Ripka et al. Arch. Biochem. Biophys. 249:533-545 (1986); Yamane-Ohnuki et al. Biotech. Bioeng. 87:614 (2004); Kanda, Y. et al., Biotechnol. Bioeng., 94 (4): 680-688 (2006)). Other antibody glycosylation variants are also included (See e.g., U.S. Pat. No. 6,602,684).

In some embodiments, an antibody provided herein has a dissociation constant (K_{D}) of about 1 μM, 100 nM, 50 nM, 40 nM, 30 nM, 20 nM, 10 nM, 5 nM, 2 nM, 1 nM, 0.5 nM, 0.1 nM, 0.05 nM, 0.01 nM or less (e.g., 10⁻⁸ M or less, e.g., from 10⁻⁸ M to 10⁻¹³ M, e.g., from 10⁻⁹ M to 10⁻¹³ M) for the antibody target. The antibody target can be an epitope target. K_{D} can be measured by any suitable assay. In certain embodiments, K_{D} can be measured using surface plasmon resonance assays (e.g., using a BIACORE®-2000, a BIACORE®-3000 or Octet).

In some embodiments, one or more amino acid modifications may be introduced into the Fc region of an antibody provided herein, thereby generating an Fc region variant. An Fc region herein is a C-terminal region of an immunoglobulin heavy chain that contains at least a portion of the constant region. An Fc region includes native sequence Fc regions and variant Fc regions. The Fc region variant may comprise a human Fc region sequence (e.g., a human IgG1, IgG2, IgG3 or IgG4 Fc region) comprising an amino acid modification (e.g., a substitution) at one or more amino acid positions.

In some embodiments, one or more amino acid modifications may be introduced into the Fc region of an antibody provided herein, thereby generating an Fc region variant. An Fc region herein is a C-terminal region of an immunoglobulin heavy chain that contains at least a portion of the constant region. An Fc region includes native sequence Fc regions and variant Fc regions. The Fc region variant may comprise a human Fc region sequence (e.g., a human IgG1, IgG2, IgG3 or IgG4 Fc region) comprising an amino acid modification (e.g., a substitution) at one or more amino acid positions.

In some instances, the Fc region of an immunoglobulin is important for many important antibody functions (e.g., effector functions), such as antigen-dependent cellular cytotoxicity (ADCC), complement dependent cytotoxicity (CDC), and antibody-dependent cell-mediated phagocytosis (ADCP), result in killing of target cells, albeit by different mechanisms. Accordingly, in some embodiments, the antibodies described herein comprise the variable domains of the invention combined with constant domains comprising different Fc regions, selected based on the biological activities of the antibody for the intended use. In certain instances, human IgGs, for example, can be classified into four subclasses, IgG1, IgG2, IgG3, and IgG4, and each these of these comprises an Fc region having a unique profile for binding to one or more of Fcγ receptors (activating receptors FcγRI (CD64), FcγRIIA, FcγRIIC (CD32); FcγRIIIA and FcγRIIIB (CD16) and inhibiting receptor FcγRIIB), and for the first component of complement (C1q). Human IgG1 and IgG3 bind to all Fcγ receptors; IgG2 binds to FcγRIIAH131, and with lower affinity to FcγRIIAR131 FcγRIIIAv158; IgG4 binds to FcγRI, FcγRIIA, FcγRIIB, FcγRIIC, and FcγRIIIAV158; and the inhibitory receptor FcγRIIB has a lower affinity for IgG1, IgG2 and IgG3 than all other Fcγ receptors. Studies have shown that FcγRI does not bind to IgG2, and FcγRIIIB does not bind to IgG2 or IgG4. Id. In general, with regard to ADCC activity, human IgG1>IgG3»IgG4>IgG2.

In some embodiments, the antibodies of this disclosure are variants that possess reduced effector functions, which make it a desirable candidate for applications in which certain effector functions (such as complement fixation and ADCC) are unnecessary or deleterious. Such antibodies can have decreased complement-dependent cytotoxicity (CDC), antibody-dependent cell cytotoxicity (ADCC), or antibody dependent cellular phagocytosis (ADCP). In some embodiments, the antibodies of this disclosure are variants that possess increased effector functions for applications in which increased immunogenicity would be beneficial. Such antibodies can have increased CDC, ADCC, or ADCP, or a combination thereof. Non-limiting examples of in vitro assays to assess ADCC activity of a molecule of interest is described in U.S. Pat. Nos. 5,500,362 and 5,821,337. Alternatively, non-radioactive assays methods may be employed (e.g., ACTI™ and CytoTox 96® non-radioactive cytotoxicity assays). Useful effector cells for such assays include peripheral blood mononuclear cells (PBMC), monocytes, macrophages, and Natural Killer (NK) cells.

Antibodies can have increased half-lives and improved binding to the neonatal Fc receptor (FcRn) (See e.g., US 2005/0014934). Such antibodies can comprise an Fc region with one or more substitutions therein which improve binding of the Fc region to FcRn, and include those with substitutions at one or more of Fc region residues: 238, 256, 265, 272, 286, 303, 305, 307, 311, 312, 317, 340, 356, 360, 362, 376, 378, 380, 382, 413, 424 or 434 according to the EU numbering system (See e.g., U.S. Pat. No. 7,371,826). Other examples of Fc region variants are also contemplated (See e.g., Duncan & Winter, Nature 322:738-40 (1988); U.S. Pat. Nos. 5,648,260; 5,624,821; and WO94/29351).

In some embodiments, it may be desirable to create cysteine engineered antibodies, e.g., “thioMAbs,” in which one or more residues of an antibody are substituted with cysteine residues. In some embodiments, the substituted residues occur at accessible sites of the antibody. Reactive thiol groups can be positioned at sites for conjugation to other moieties, such as drug moieties or linker drug moieties, to create an immunoconjugate. In some embodiments, any one or more of the following residues may be substituted with cysteine: V205 (Kabat numbering) of the light chain; A118 (EU numbering) of the heavy chain; and S400 (EU numbering) of the heavy chain Fc region.

In some embodiments, an antibody provided herein may be further modified to contain additional nonproteinaceous moieties that are known and available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n vinyl pyrrolidone) polyethylene glycol, polypropylene glycol homopolymers, polypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if two or more polymers are attached, they can be the same or different molecules.

This disclosure also provides for immunoconjugates comprising an anti-NR1 subunit antibody described herein. An immunoconjugate is an antibody conjugated to one or more heterologous molecule(s). For example, an immunoconjugate can comprise an anti-NR1 subunit antibody conjugated to one or more cytotoxic agents, such as chemotherapeutic agents or drugs, growth inhibitory agents, protein domains, toxins (e.g., protein toxins, enzymatically active toxins of bacterial, fungal, plant, or animal origin, or fragments thereof), or radioactive isotopes. In some embodiments, an immunoconjugate can comprise an anti-NR1 antibody, or fragment thereof (e.g., an scFv).

The antibodies described herein can be encoded by a nucleic acid. A nucleic acid is a type of polynucleotide comprising two or more nucleotide bases. In certain embodiments, the nucleic acid is a component of a vector that can be used to transfer the polypeptide encoding polynucleotide into a cell. As used herein, the term “vector” refers to a nucleic acid molecule capable of transporting another nucleic acid to which it has been linked. One type of vector is a genomic integrated vector, or “integrated vector,” which can become integrated into the chromosomal DNA of the host cell. Another type of vector is an “episomal” vector, e.g., a nucleic acid capable of extra-chromosomal replication. Vectors capable of directing the expression of genes to which they are operatively linked are referred to herein as “expression vectors.” Suitable vectors comprise plasmids, bacterial artificial chromosomes, yeast artificial chromosomes, viral vectors, and the like. In the expression vectors regulatory elements such as promoters, enhancers, polyadenylation signals for use in controlling transcription can be derived from mammalian, microbial, viral, or insect genes. The ability to replicate in a host, usually conferred by an origin of replication, and a selection gene to facilitate recognition of transformants may additionally be incorporated. Vectors derived from viruses, such as lentiviruses, retroviruses, adenoviruses, adeno-associated viruses, and the like, may be employed. Plasmid vectors can be linearized for integration into a genomic region. In certain embodiments, the expression vector is a plasmid. In certain embodiments, the expression vector is a lentivirus, adenovirus, or adeno-associated virus. In certain embodiments, the expression vector is an adenovirus. In certain embodiments, the expression vector is an adeno-associated virus. In certain embodiments, the expression vector is a lentivirus.

As used herein, the terms “homologous,” “homology,” or “percent homology” when used herein to describe to an amino acid sequence or a nucleic acid sequence, relative to a reference sequence, can be determined using the formula described by Karlin and Altschul (Proc. Natl. Acad. Sci. USA 87:2264-2268, 1990, modified as in Proc. Natl. Acad. Sci. USA 90:5873-5877, 1993). Such a formula is incorporated into the basic local alignment search tool (BLAST) programs of Altschul et al. (J. Mol. Biol. 215:403-410, 1990). Percent homology of sequences can be determined using the most recent version of BLAST, as of the filing date of this application.

The nucleic acids encoding the antibodies described herein can be used to infect, transfect, transform, or otherwise render a suitable cell transgenic for the nucleic acid, thus enabling the production of antibodies for commercial or therapeutic uses. Standard cell lines and methods for the production of antibodies from a large-scale cell culture are known in the art. See e.g., Li et al., “Cell culture processes for monoclonal antibody production.” Mabs. 2010 September-Oct; 2 (5): 466-477. In certain embodiments, the cell is a Eukaryotic cell. In certain embodiments, the Eukaryotic cell is a mammalian cell. In certain embodiments, the mammalian cell is a cell line useful for producing antibodies is a Chines Hamster Ovary cell (CHO) cell, an NSO murine myeloma cell, or a PER.C6® cell. In certain embodiments, the nucleic acid encoding the antibody is integrated into a genomic locus of a cell useful for producing antibodies. In certain embodiments, described herein is a method of making an antibody comprising culturing a cell comprising a nucleic acid encoding an antibody under conditions in vitro sufficient to allow production and secretion of said antibody.

In certain embodiments, described herein, is a master cell bank comprising: (a) a mammalian cell line comprising a nucleic acid encoding an antibody described herein integrated at a genomic location; and (b) a cryoprotectant. In certain embodiments, the cryoprotectant comprises glycerol or DMSO. In certain embodiments, the master cell bank comprises: (a) a CHO cell line comprising a nucleic acid encoding an antibody with (i) a heavy chain amino acid sequence set forth by any one of SEQ ID NOs: 1, 3, 5, 7, or 9; and (ii) a light chain amino acid sequence set forth by any one of SEQ ID NOs: 2, 4, 6, 8, or 10 integrated at a genomic location; and (b) a cryoprotectant. In certain embodiments, the cryoprotectant comprises glycerol or DMSO. In certain embodiments the CHO cell line comprises a nucleic acid with any one of SEQ ID NOs: 42-51 integrated at a genomic location. In certain embodiments, the master cell bank is contained in a suitable vial or container able to withstand freezing by liquid nitrogen.

Also described herein are methods of making an antibody described herein. Such methods comprise incubating a cell or cell-line comprising a nucleic acid encoding the antibody in a cell culture medium under conditions sufficient to allow for expression and secretion of the antibody, and further harvesting the antibody from the cell culture medium. The harvesting can further comprise one or more purification steps to remove live cells, cellular debris, non-antibody proteins or polypeptides, undesired salts, buffers, and medium components. In certain embodiments, the additional purification step(s) include centrifugation, ultracentrifugation, protein A, protein G, protein A/G, or protein L purification, and/or ion exchange chromatography.

“Treat,” “treatment,” or “treating,” as used herein refers to, e.g., a deliberate intervention to a physiological disease state resulting in the reduction in severity of a disease or condition; the reduction in the duration of a condition course; the amelioration or elimination of one or more symptoms associated with a disease or condition; or the provision of beneficial effects to a subject with a disease or condition. Treatment does not require curing the underlying disease or condition.

A “therapeutically effective amount,” “effective dose,” “effective amount,” or “therapeutically effective dosage” of a drug or therapeutic agent is any amount of the drug that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

As used herein, “pharmaceutically acceptable” with reference to a carrier” “excipient” or “diluent” includes any and all solvents, dispersion media, coatings, antibacterial and antifungal agents, isotonic and absorption delaying agents, and the like that are physiologically compatible. In some aspects, the carrier is suitable for intravenous, intramuscular, subcutaneous, parenteral, spinal or epidermal administration (e.g., by injection or infusion). Depending on the route of administration, the active compound, i.e., antibody, can be coated in a material to protect the compound from the action of acids and other natural conditions that can inactivate the compound.

The pharmaceutical compounds described herein can include one or more pharmaceutically acceptable salts. A “pharmaceutically acceptable salt” refers to a salt that retains the desired biological activity of the parent compound and does not impart any undesired toxicological effects (see e.g., Berge, S. M., et al. (1977) J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono-and dicarboxylic acids, phenyl-substituted alkanoic acids, hydroxy alkanoic acids, aromatic acids, aliphatic and aromatic sulfonic acids, and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

### Therapeutic Methods

In certain embodiments, disclosed herein, are antibodies useful for the treatment of a psychiatric or central nervous system disorder or disease. Treatment refers to a method that seeks to improve or ameliorate the condition being treated. With respect to psychiatric or central nervous system disease or conditions, treatment includes, but is not limited to, reduction of one or more symptoms of a psychiatric or central nervous system disorder. In certain embodiments, treatment will affect severity of a psychiatric or central nervous systems disease or disorder. In certain embodiments, treatment encompasses use as a prophylactic or maintenance dose intended to prevent reoccurrence or progression of a previously treated psychiatric or central nervous system disease or disorder. It is understood by those of skill in the art that not all individuals will respond equally or at all to a treatment that is administered, nevertheless these individuals are considered to be treated.

In some embodiments, the symptoms of a psychiatric or central nervous system disease or disorder can include seizures, delusional thought, speech disorders, movement difficulty, or aggression.

In certain embodiments, the psychiatric or central nervous system disease or disorder comprises anti-NMDAR encephalitis, schizophrenia, psychosis, bipolar disorder, depression, epilepsy, or dementia. In certain embodiments, the psychiatric or central nervous system disease or disorder is a N-methyl-D-aspartate (NMDA) receptor dysfunction. In a certain embodiment, the psychiatric or central nervous system disease or disorder treated is relapsed.

In certain embodiments, the methods described herein comprise determining in a biological sample from an individual if said individual has a pathogenic organism reactive antibody, and then treating the individual with the pathogen associated autoantibody with an antibody or antigen binding fragment thereof binds a NMDAR1 (NR1) subunit of the NMDA receptor. In certain embodiments, exemplary pathogenic organisms are listed in Table 3.

In certain embodiments, the individual displays one or more symptoms associated with schizophrenia. In certain embodiments, the symptom associated with schizophrenia comprises one or more of delusions, hallucinations, disorganized speech, or abnormal motor behavior. In certain embodiments, the individual has been diagnosed with schizophrenia. In certain embodiments, the individual is diagnosed as having schizophrenia based on the Brief Clinical Assessment Scale for Schizophrenia (BCASS) or the Positive and Negative Syndrome Scale for Schizophrenia (PANSS).

In some embodiments, clinical improvement before and/or after treatment is evaluated using the BCASS, with improvement in BCASS scores obtained after treatment. In some embodiments, after treatment, BCASS s reduced by 90% or more. In some embodiments, after treatment, BCASS is reduced by 80% or more. In some embodiments, after treatment, BCASS is reduced by 70% or more. In some embodiments, after treatment, BCASS is reduced by 60% or more. In some embodiments, after treatment, BCASS is reduced by 50% or more. In some embodiments, after treatment, BCASS is reduced by 40% or more. In some embodiments, after treatment, BCASS total score is by 30% or more. In some embodiments, after treatment, BCASS is reduced by 20% or more. In some embodiments, after treatment, BCASS is reduced by 10% or more. In some embodiments, after treatment, BCASS is reduced by 5% or more.

In some embodiments, clinical improvement before and/or after treatment is evaluated using the PANSS, with improvement in PANSS scores obtained after treatment. In some embodiments, after treatment, PANSS s reduced by 90% or more. In some embodiments, after treatment, PANSS is reduced by 80% or more. In some embodiments, after treatment, PANSS is reduced by 70% or more. In some embodiments, after treatment, PANSS is reduced by 60% or more. In some embodiments, after treatment, PANSS is reduced by 50% or more. In some embodiments, after treatment, PANSS is reduced by 40% or more. In some embodiments, after treatment, PANSS total score is by 30% or more. In some embodiments, after treatment, PANSS is reduced by 20% or more. In some embodiments, after treatment, PANSS is reduced by 10% or more. In some embodiments, after treatment, PANSS is reduced by 5% or more.

In certain embodiments, the individual displays one or more symptoms associated with bipolar disorder. In certain embodiments, the symptom associated with bipolar disorder comprises one or more of increased energy, excitement, impulsive behavior, agitation, lack of energy, feeling worthless, low self-esteem or suicidal thoughts. In certain embodiments, the individual has been diagnosed with bipolar disorder. In certain embodiments, the individual is diagnosed as having bipolar disorder based on the Bipolar Depression Rating Scale (BDRS) or Bipolar Spectrum Diagnostic Scale (BSDS).

In some embodiments, clinical improvement before and/or after treatment is evaluated using the BDRS, with improvement in BDRS scores obtained after treatment. In some embodiments, after treatment, BDRS is 50 or lower. In some embodiments, after treatment, BDRS is 40 or lower. In some embodiments, after treatment, BDRS is 30 or lower. In some embodiments, after treatment, BDRS is 20 or lower. In some embodiments, after treatment, BDRS is 10 or lower. In some embodiments, after treatment, BDRS is 5 or lower. In some embodiments, after treatment, BDRS is 0. In some embodiments, an individual receiving treatment will score 1, 10, 20, 30, 40, or 50 points lower compared to a pretreatment assessment.

In some embodiments, clinical improvement before and/or after treatment is evaluated using the BSDS, with improvement in BSDS scores obtained after treatment. In some embodiments, after treatment, BSDS is 20 or lower. In some embodiments, after treatment, BSDS is 15 or lower. In some embodiments, after treatment, BSDS is 10 or lower. In some embodiments, after treatment, BSDS is 5 or lower. In some embodiments, after treatment, BSDS is 0. In some embodiments, an individual receiving treatment will score 1, 5, 10, 15, 20, or 25 points lower compared to a pretreatment assessment.

In certain embodiments, the antibody or antigen binding fragment thereof binds a NMDAR1 (NR1) subunit of the NMDA receptor binds to one or more amino acid residues comprised within the amino acid sequence LQNRKLV (SEQ ID NO: 41). In certain embodiments, the antibody or antigen binding fragment thereof binds a NMDAR1 (NR1) subunit of the NMDA receptor is any one or more of those disclosed in Table 1 of this disclosure.

In certain embodiments, the methods described herein are not for treatment of autoimmune encephalitis. In certain embodiments, the methods described herein are not for treatment of NMDAR associated autoimmune encephalitis.

In certain embodiments, the antibodies can be administered to a subject in need thereof by any route suitable for the administration of antibody-containing pharmaceutical compositions, such as, for example, subcutaneous, intraperitoneal, intravenous, intramuscular, intratumoral, or intracerebral, etc. In certain embodiments, the antibodies are administered intravenously. In certain embodiments, the antibodies are administered subcutaneously. In certain embodiments, the antibodies are administered intratumoral. In certain embodiments, the antibodies are administered on a suitable dosage schedule, for example, weekly, twice weekly, monthly, twice monthly, once every two weeks, once every three weeks, or once a month etc. In certain embodiments, the antibodies are administered once every three weeks. The antibodies can be administered in any therapeutically effective amount. In certain embodiments, the therapeutically acceptable amount is greater than about 50 mg/kg, 75 mg/kg, 100 mg/kg, 125 mg/kg, 150 mg/kg, 175 mg/kg, and 200 mg/kg. In certain embodiments, the therapeutically acceptable amount is between about 0.1 mg/kg and about 200 mg/kg. In certain embodiments, the therapeutically acceptable amount is between about 1 mg/kg and about 40 mg/kg. In certain embodiments, the therapeutically acceptable amount is between about 1 mg/kg and about 20 mg/kg. In certain embodiments, the therapeutically acceptable amount is between about 1 mg/kg and about 10 mg/kg. In certain embodiments, the therapeutically acceptable amount is between about 5 mg/kg and about 30 mg/kg. In certain embodiments, the therapeutically acceptable amount is between about 5 mg/kg and about 20 mg/kg. Therapeutically effective amounts include amounts sufficient to ameliorate one or more symptoms associated with the disease or affliction to be treated.

### Pharmaceutically Acceptable Excipients, Carriers, and Diluents

In certain embodiments the anti-NR1 NMDA receptor subunit antibodies of the current disclosure are included in a pharmaceutical composition comprising one or more pharmaceutically acceptable excipients, carriers, and diluents. Pharmaceutically acceptable excipients, carriers and diluents can be included to increase shelf-life, stability, or the administrability of the antibody. Such compounds include salts, pH buffers, detergents, anti-coagulants, and preservatives. In certain embodiments, the antibodies of the current disclosure are administered suspended in a sterile solution. In certain embodiments, the solution comprises about 0.9% NaCl. In certain embodiments, the solution comprises about 5.0% dextrose. In certain embodiments, the solution further comprises one or more of: buffers, for example, acetate, citrate, histidine, succinate, phosphate, bicarbonate and hydroxymethylaminomethane (Tris); surfactants, for example, polysorbate 80 (Tween 80), polysorbate 20 (Tween 20), and poloxamer 188; polyol/disaccharide/polysaccharides, for example, glucose, dextrose, mannose, mannitol, sorbitol, sucrose, trehalose, and dextran 40; amino acids, for example, glycine or arginine; antioxidants, for example, ascorbic acid, methionine; or chelating agents, for example, EDTA or EGTA.

In certain embodiments, the antibodies of the current disclosure can be shipped/stored lyophilized and reconstituted before administration. In certain embodiments, lyophilized antibody formulations comprise a bulking agent such as, mannitol, sorbitol, sucrose, trehalose, dextran 40, or combinations thereof. The lyophilized formulation can be contained in a vial comprised of glass or other suitable non-reactive material. The antibodies when formulated, whether reconstituted or not, can be buffered at a certain pH, generally less than 7.0. In certain embodiments, the pH can be between 4.5 and 7.0, 4.5 and 6.5, 4.5 and 6.0, 4.5 and 5.5, 4.5 and 5.0, or 5.0 and 6.0.

Also described herein are kits comprising one or more of the antibodies described herein in a suitable container and one or more additional components selected from: instructions for use; a diluent, an excipient, a carrier, and a device for administration.

In certain embodiments, described herein is a method of preparing a psychiatric or central nervous system disorder or disease treatment comprising admixing one or more pharmaceutically acceptable excipients, carriers, or diluents and an antibody of the current disclosure. In certain embodiments, described herein is a method of preparing a cancer treatment for storage or shipping comprising lyophilizing one or more antibodies of the current disclosure.

The foregoing descriptions of specific embodiments of the present disclosure have been presented for purposes of illustration and description. They are not intended to be exhaustive or to limit the invention to the precise forms disclosed, and obviously many modifications and variations are possible in light of the above teaching. The embodiments were chosen and described to best explain the principles of the invention and its practical application, to thereby enable others skilled in the art to best utilize the present disclosure and various embodiments with various modifications as are suited to the particular use contemplated. It is intended that the scope of the invention be defined by the Claims appended hereto and their equivalents.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### EXAMPLES

The following illustrative examples are representative of embodiments of compositions and methods described herein and are not meant to be limiting in any way.

### Example 1: The Predicted Epitope of the NMDA Receptor is LQNRKLV

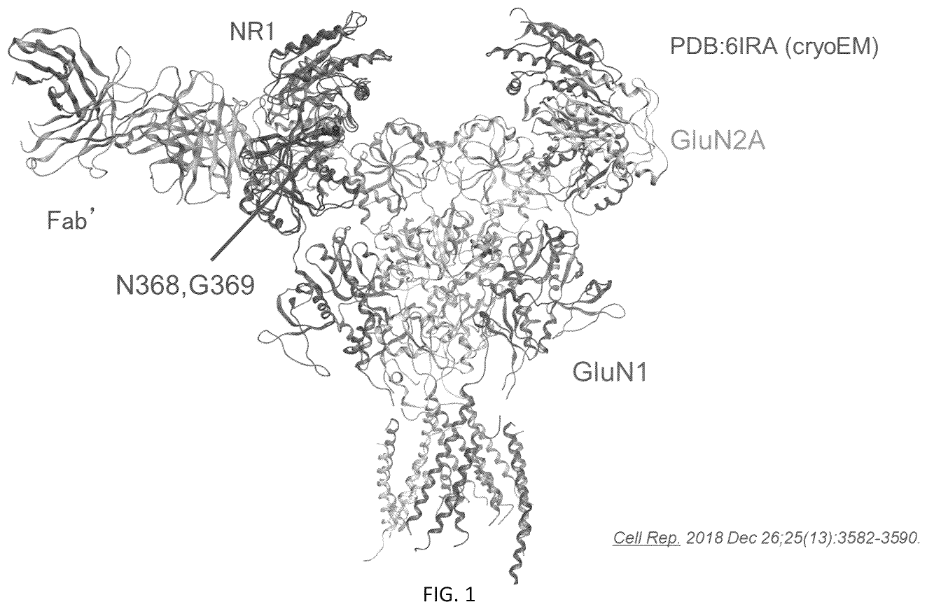

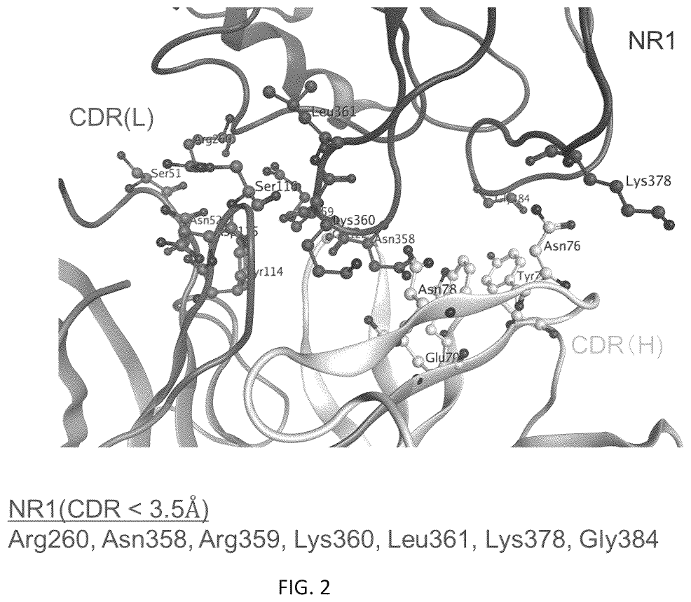

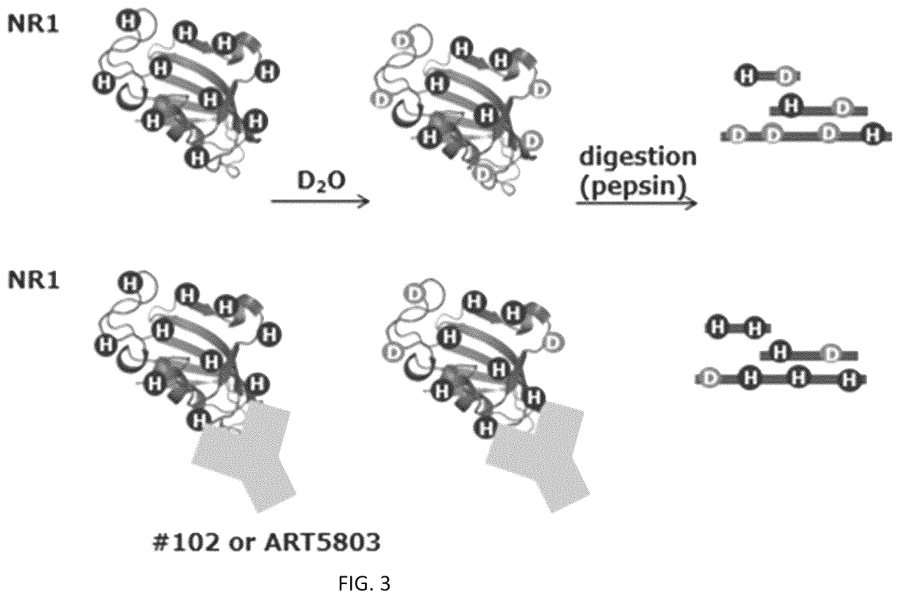

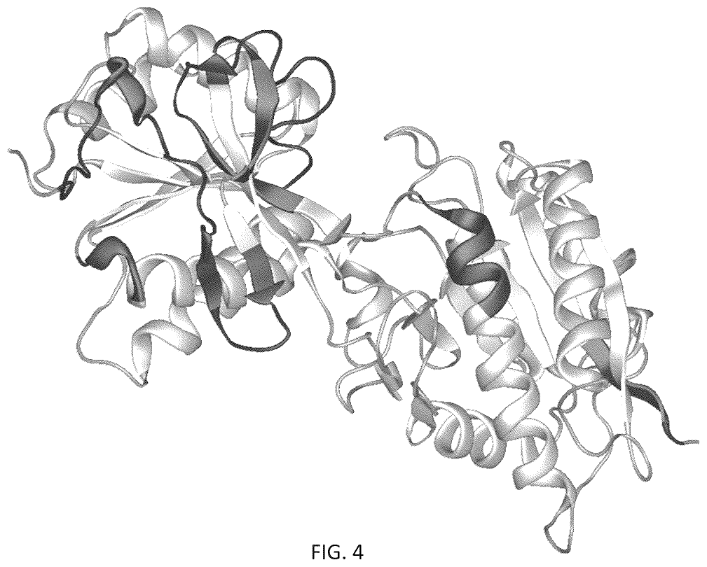

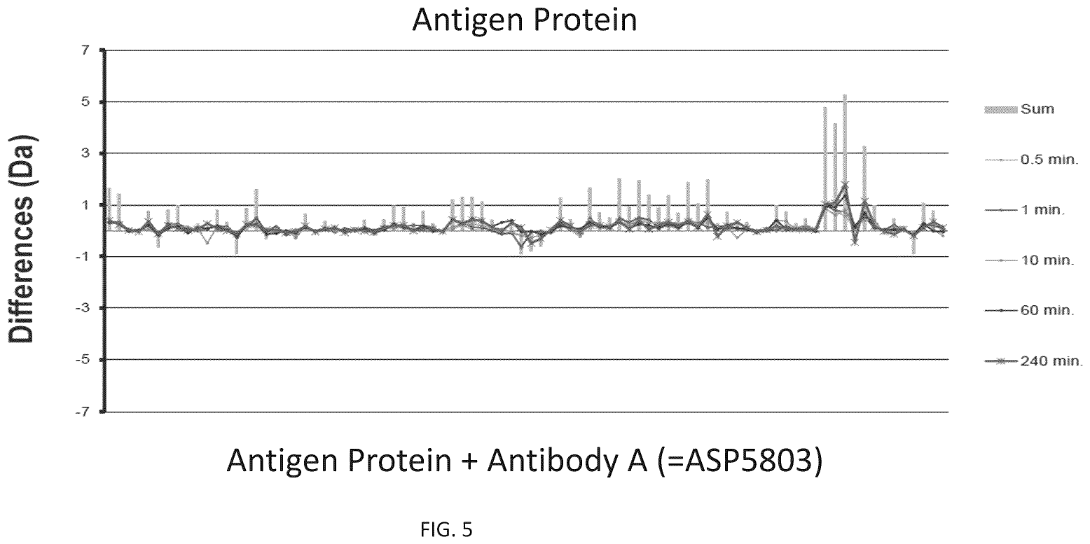

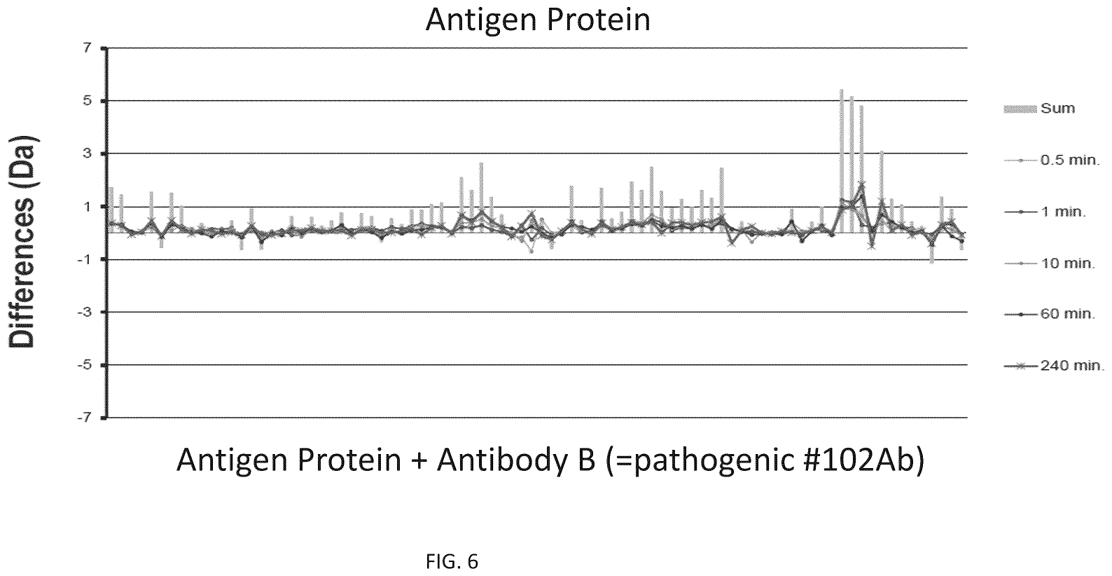

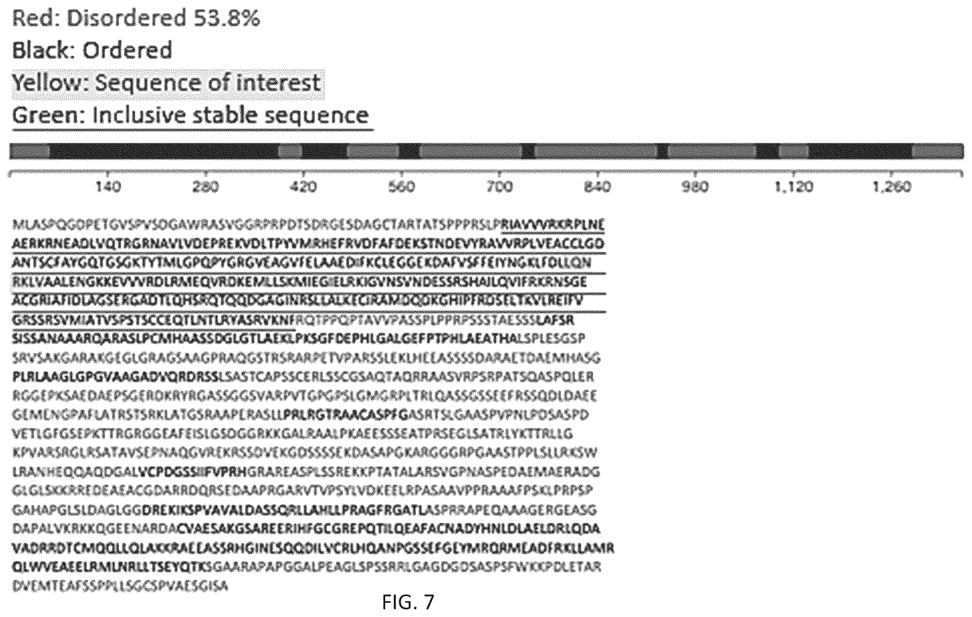

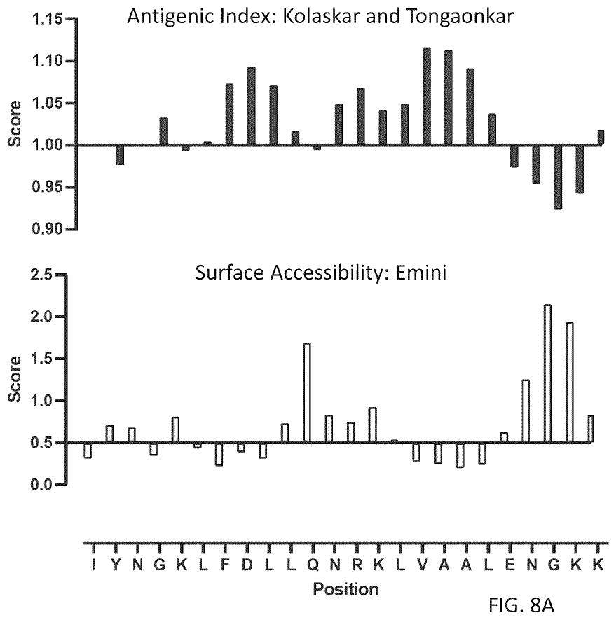

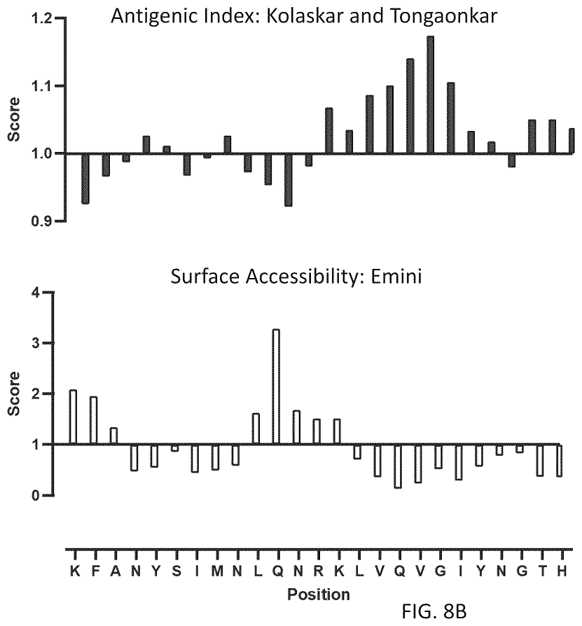

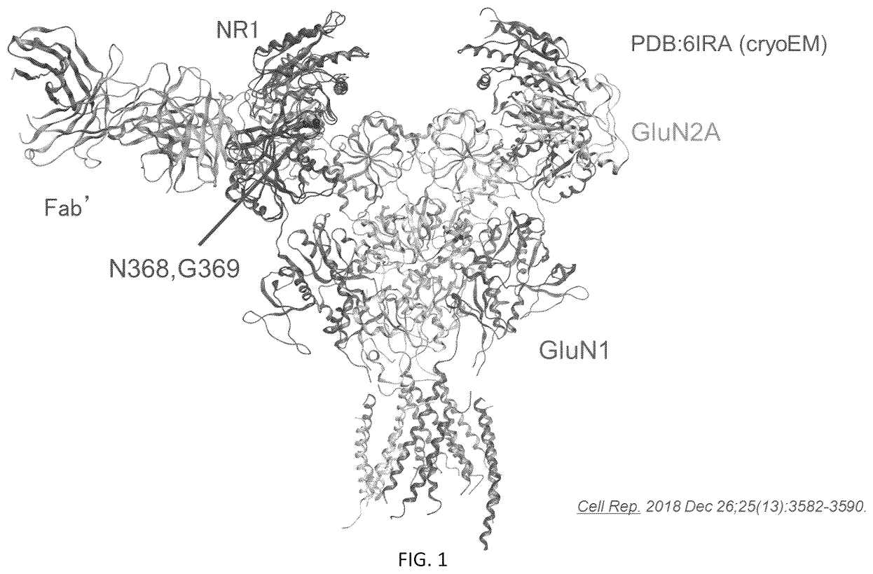

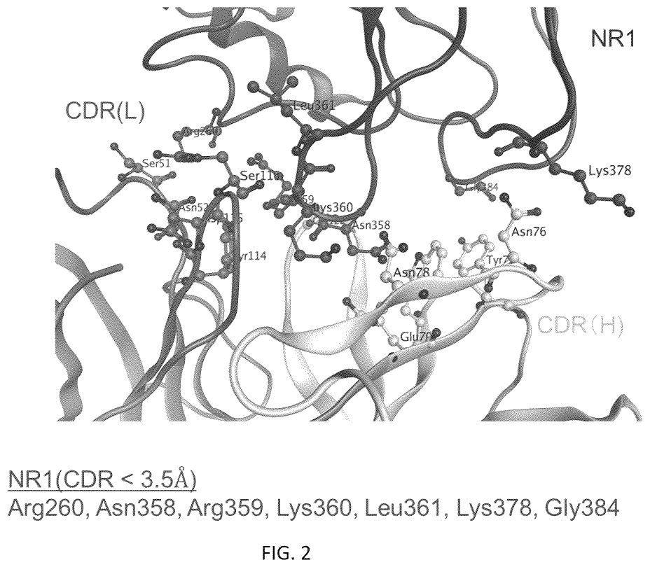

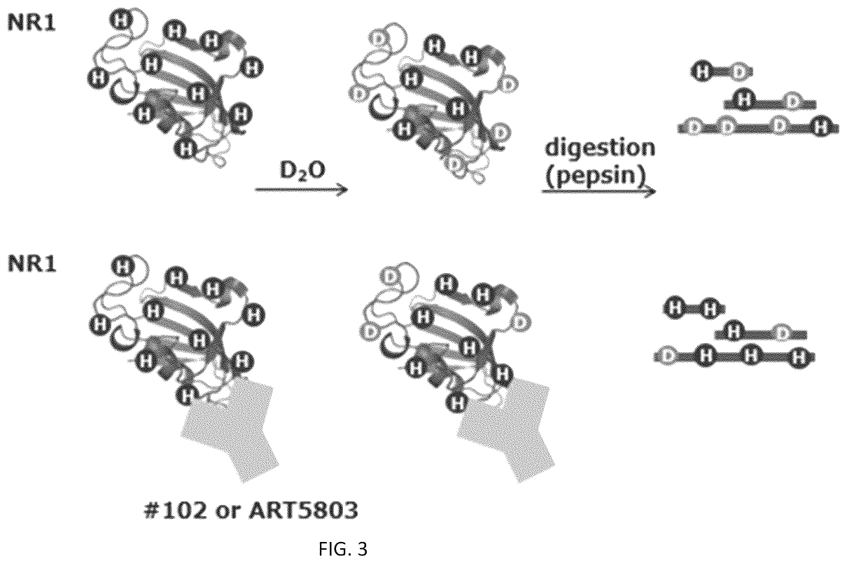

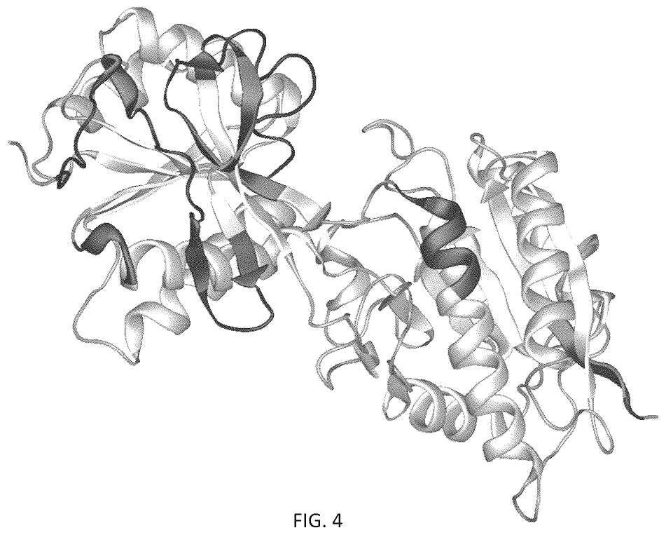

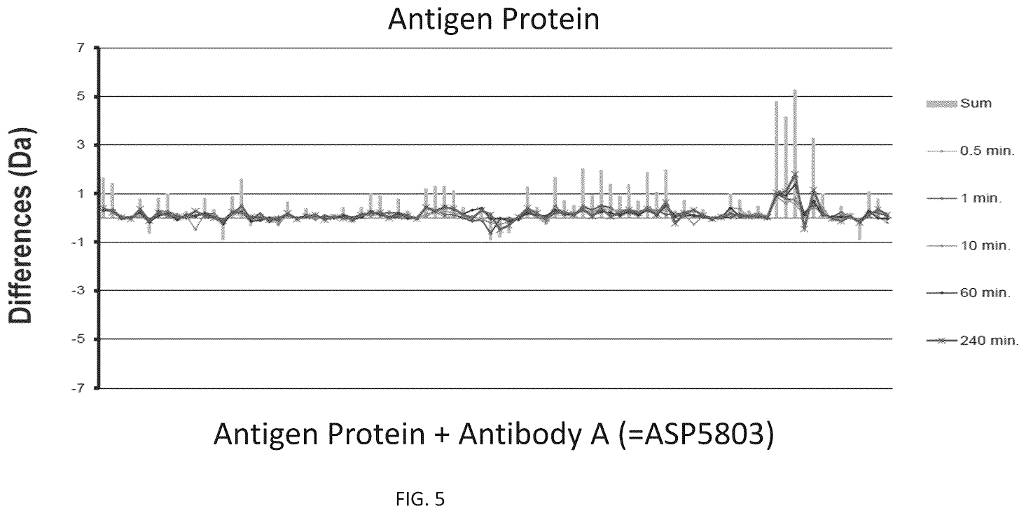

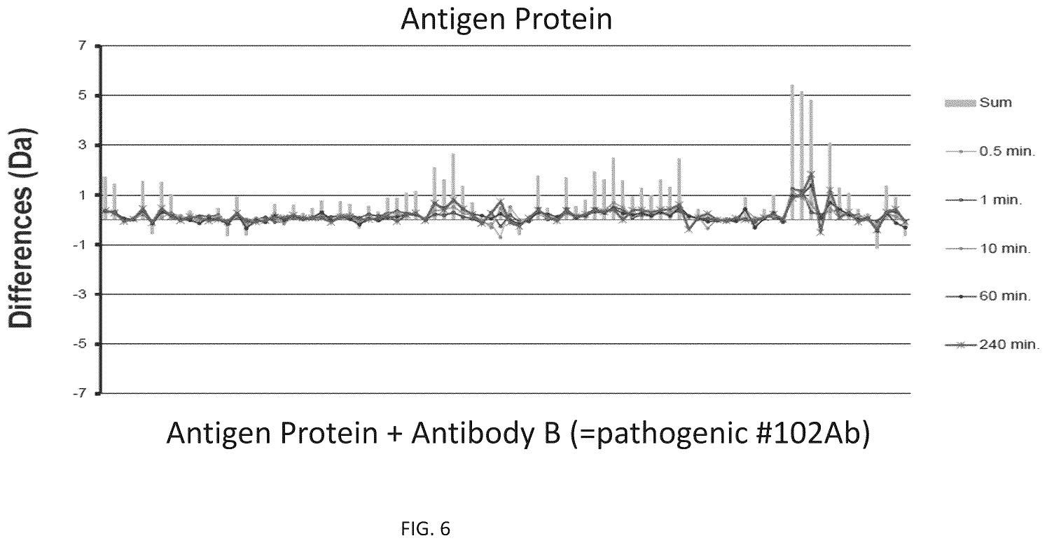

X-ray crystallography (FIGS. 1, 2) and Hydrogen Deuterium Exchange Mass Spectrometry (HDX-MS) (FIG. 3, 4, 5, 6) were performed on the binding complex of an autoantibody and a therapeutic antibody, respectively, specific to the NR1 subunit of the NMDA receptor to identify the binding epitope of the NR1 subunit. X-ray crystallography: A pathogenic autoantibody cloned from an anti-NMDA receptor encephalitis patient (#102Ab) Fab′ and NMDA receptor NR1 Amino-Terminal Domain (ATD) were used. The crystal structure of NMDA receptor NR1 ATD in complex with the #102Ab at a resolution of 3.5 Å. HDX-MS: A therapeutic one-armed antibody (ASP5803) or #102Ab and NMDA receptor NR1 ATD were used. The unbound NR1 ATD and antibody-bound NR1 ATD were incubated in deuterated (D20) water in order to exchange any amide hydrogens with deuterium from exposed amino acids of the protein's backbone. The location of these deuterium molecules on the proteins sequence were determined using high resolution mass spectrometry. The predicted epitopes, verified by both HDX-MS and X-ray crystallography, can be seen in Table 2.

TABLE 2
NR1 binding region identified by
HDX-MS and X-Ray Crystallography

| | | | X-Ray Crystallography | |
|---|---|---|---|---|
| HDX-MS | | | | Ab#102-NR1 |
| Fragment | ASP5803 | Ab #102 | Fragment | (CDR < 3.5 Å) |
| 1 | K25-V27 | K25-V27 | 1 | |
| | | V46 | 2 | |
| | | K51-H53 | 3 | |
| 2 | V157-N161 | V157-N161 | 4 | |
| | | A211-A216 | 5 | |
| 3 | E251-L269 | E251-L269 | 6 | Arg260 |
| 4 | N276-S282 | N276-S282 | 7 | |
| 5 | Q357-Q363 | Q357-Q363 | 8 | Asn358, Arg359, |
| | | | | Lys360, Leu361 |
| 6 | I366-G391 | I366-G391 | 9 | Lys378, Gly384 |

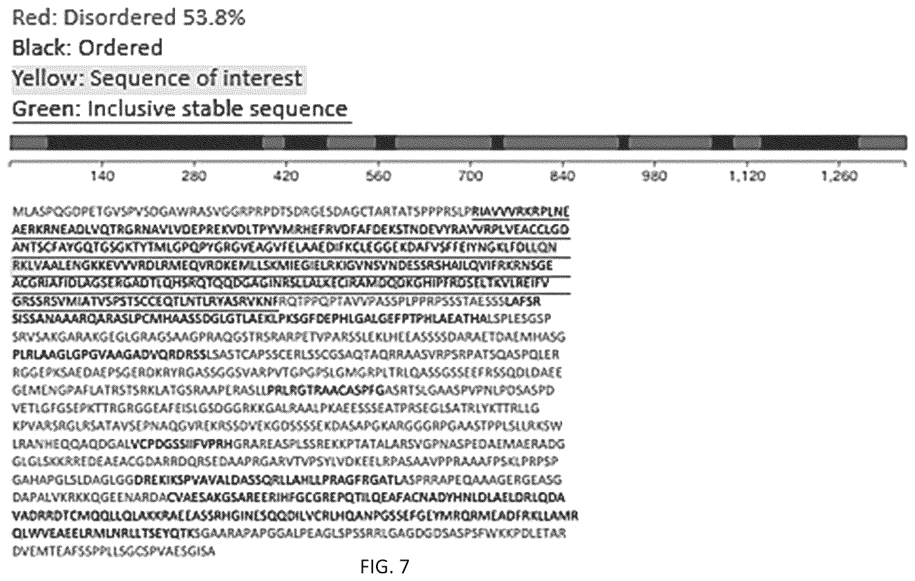

The identified epitope, “LQNRKLV” was then identified in the sequence of the whole internal kinesin motor domain of Toxoplasma gondii (FIG. 7, SEQ ID NO: 42).

The sequence of the whole internal kinesin motor domain of Toxoplasma gondii (T. gondii):

(SEQ ID NO: 42)
MLASPQGDPETGVSPVSDGAWRASVGGRPRPDTSDRGESDAGCTARTAT
SPPPRSLPRIAVVVRKRPLNEAERKRNEADLVQTRGRNAVLVDEPREKV
DLTPYVMRHEFRVDFAFDEKSTNDEVYRAVVRPLVEACCLGDANTSCFA
YGQTGSGKTYTMLGPQPYGRGVEAGVFELAAEDIFKCLEGGEKDAFVSF
FEIYNGKLFDLLQNRKLVAALENGKKEVVVRDLRMEQVRDKEMLLSKMI
EGIELRKIGVNSVNDESSRSHAILQVIFRKRNSGEACGRIAFIDLAGSE
RGADTLQHSRQTQQDGAGINRSLLALKECIRAMDQDKGHIPFRDSELTK
VLREIFVGRSSRSVMIATVSPSTSCCEQTLNTLRYASRVKNFRQTPPQP
TAVVPASSPLPPRPSSSTAESSSLAFSRSISSANAAARQARASLPCMHA
ASSDGLGTLAEKLPKSGFDEPHLGALGEFPTPHLAEATHALSPLESGSP
SRVSAKGARAKGEGLGRAGSAAGPRAQGSTRSRARPETVPARSSLEKLH
EEASSSSDARAETDAEMHASGPLRLAAGLGPGVAAGADVQRDRSSLSAS
TCAPSSCERLSSCGSAQTAQRRAASVRPSRPATSQASPQLERRGGEPKS
AEDAEPSGERDKRYRGASSGGSVARPVTGPGPSLGMGRPLTRLQASSGS
SEEFRSSQDLDAEEGEMENGPAFLATRSTSRKLATGSRAAPERASLLPR
LRGTRAACASPFGASRTSLGAASPVPNLPDSASPDVETLGFGSEPKTTR
GRGGEAFEISLGSDGGRKKGALRAALPKAEESSSEATPRSEGLSATRLY
KTTRLLGKPVARSRGLRSATAVSEPNAQGVREKRSSDVEKGDSSSSEKD
ASAPGKARGGGRPGAASTPPLSLLRKSWLRANHEQQAQDGALVCPDGSS
IIFVPRHGRAREASPLSSREKKPTATALARSVGPNASPEDAEMAERADG
GLGLSKKRREDEAEACGDARRDQRSEDAAPRGARVTVPSYLVDKEELRP
ASAAVPPRAAAFPSKLPRPSPGAHAPGLSLDAGLGGDREKIKSPVAVAL
DASSQRLLAHLLPRAGFRGATLASPRRAPEQAAAGERGEASGDAPALVK
RKKQGEENARDACVAESAKGSAREERIHFGCGREPQTILQEAFACNADY
HNLDLAELDRLQDAVADRRDTCMQQLLQLAKKRAEEASSRHGINESQQD
ILVCRLHQANPGSSEFGEYMRQRMEADFRKLLAMRQLWVEAEELRMLNR
LLTSEYQTKSGAARAPAPGGALPEAGLSPSSRRLGAGDGDSASPSFWKK
PDLETARDVEMTEAFSSPPLLSGCSPVAESGISA.

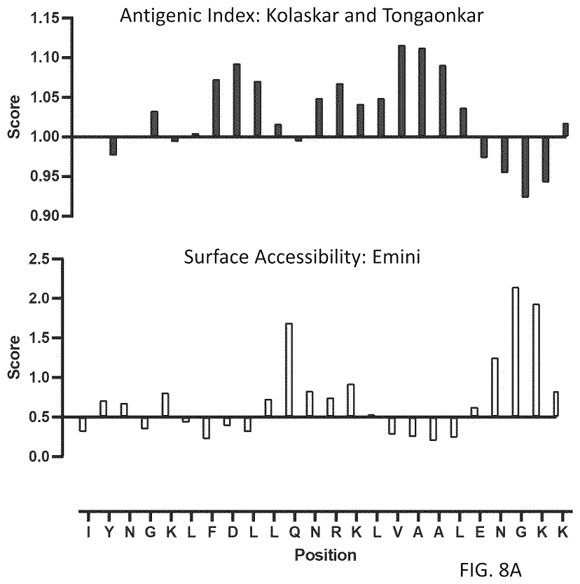

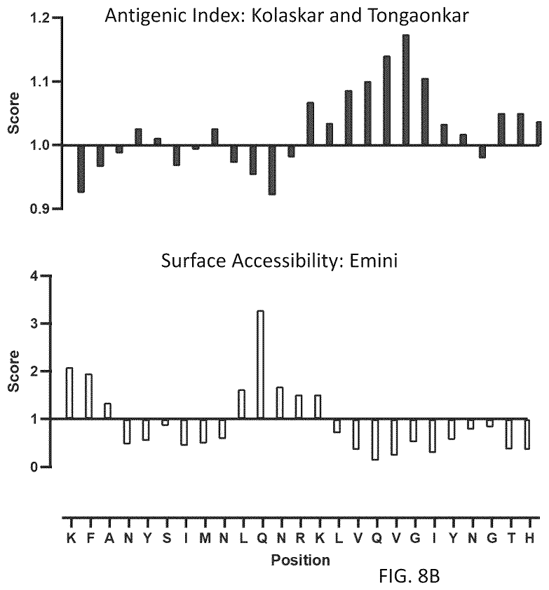

As can be seen in FIGS. 8A and 8B, the “LQNRKLV” peptide was found in NMDA receptor NR1 and the internal kinesin motor domain of T. gondii.

As summarized in the Table 3, the “LQNRKLV” peptide was found in proteins encoded in parasite or bacteria related to human neuropsychiatric disorders.

TABLE 3
Pathogenic species with homologous sequences to NMDA receptor subunits
(100% epitope match only, with at least one literature supporting (at
genus level) any psychiatric, CNS, or direct neurological implications)

| | Genus | Species | Sequence ID | Protein ID |
|---|---|---|---|---|
| Parasite | Toxoplasma | gondii | multi strain | internal kinesin motor |
| | | | | domain protein |
| | Paramecium | pentaurelia | CAD8173539.1 | unnamed protein product |
| | | tetraurelia | XP_001433531.1 | uncharacterized protein |
| | | | | GSPATT00035263001 |
| | | | XP_001450091.1 | uncharacterized protein |
| | | | | GSPATT00017065001 |
| | | octaurelia | CAD8157662.1 | unnamed protein product |
| | | | CAD8213323.1 | unnamed protein product |
| | | primaurelia | CAD8075787.1 | unnamed protein product |
| Bacteria | Campylobacter | sonneborni | CAD8123220.1 | unnamed protein product |
| | | pelordis | WP_147575871.1 | inverse autotransporter |
| | | | | beta domain-containing |
| | | | | protein |
| | | ornithocola | QKF57284.1 | hypothetical protein |
| | | | | CORN_0763 |
| | | | WP_066008439.1 | tetratricopeptide repeat |
| | | | | protein |
| | | Iari | WP_257397452.1 | hypothetical protein |
| | | | EAK0494263.1 | hypothetical protein |
| | Enterococcus | caccae | WP_010772549.1 | DNA primase |
| | Peptoniphilus | ING2-D1G | CDZ75713.1 | hemolysin |
| | | catoniae | WP_138160600.1 | MaIY/PatB family protein |
| | Paenalcaligenes | suwonensis | WP_166409601.1 | LysR family |
| | | | | transcriptional regulator |
| | Pseudomonas | multispecies | MAP30115.1 | 2,5-didehydrogluconate |
| | | | | reductase DkgB |
| | | viridiflava | WP_122808001.1 | PAS domain-containing |
| | | | | methyl-accepting |
| | | | | chemotaxis protein |
| | | mendocina NK-01 | AEB56118.1 | aldo/keto reductase |
| | Burkholderia | multispecies | WP_166913548.1 | 2,5-didehydrogluconate |
| | | | | reductase DkgB |
| | | multivorans CGD2 | EEE05529.1 | 2,5-diketo-D-gluconic acid |
| | | | | reductase A |
| | | multivorans CGD1 | EEE01682.1 | 2,5-diketo-D-gluconic acid |
| | | multivorans CGD2 | EEE05529.1 | reductase B |
| | | cenocepacia K56- | EPZ89645.1 | |
| | | 2Valvano | | |
| | | diffusa | CAG9252122.1 | methylglyoxal reductase |
| | | cenocepacia | CAD9221280.1 | DkgB |
| | | cenocepacia | SOT44821.1 | Aldehyde reductase |
| | | dolosa AU0158 | EAY71159.1 | NADP dependent sorbitol |
| | | | | 6-phosphate |
| | | | | dehydrogenase |
| | | multivorans CF2 | EJO61145.1 | oxidoreductase, aldo/keto |
| | | | | reductase family protein |
| | Chromobacterium | multispecies | WP_118268538.1 | 2,5-didehydrogluconate |
| | | | | reductase DkgB |
| | Acinetobacter | baumannii | WP_114159739.1 | LysR family |
| | | calcoaceticus/baumanni | WP_001163442.1 | transcriptional regulator |
| | | complex | | |
| | | nosocomialis | WP_104918929.1 | |
| | | baumannii | WP_162283112.1 | LysR substrate-binding |
| | | | | domain-containing protein |
| | | A47 | WP_038346411.1 | 2,5-didehydrogluconate |
| | | S40 | WP_200458926.1 | reductase DkgB |
| | | unclassified, | WP_200482793.1 | |
| | | multispecies | | |
| | | ANC3789 | WP_004749200.1 | |
| | Paenibacillus | physcomitrellae | WP_094095067.1 | Gfo/ldh/MocA family |
| | | | | oxidoreductase |
| | Escherichia | coli | EFA4879647.1 | EscU/YscU/HrcU family |
| | | marmotae | WP_038355821. 1 | type III secretion export |
| | | | | apparatus switch protein |
| | | coli | WP_224219376.1 | 2,5-didehydrogluconate |
| | | coli | EET3164582.1 | reductase DkgB |
| | Nocardia | multispecies | WP_040703631.1 | hypothetical protein |

### Example 2: Immunization of Toxoplasma/Bacterial Proteins to Induce Anti-NMDA Receptor Antibodies in Mice

Mice (Balb/c) are immunized with toxoplasma or parasite/bacterial proteins sharing 100% amino acid sequence (LQNRKLV) with the NR1 subunit of the NMDA receptor shown in Table 3 (25 μg emulsion) with TiterMax Gold by four biweekly intradermal injections. Two weeks after, antigen protein (25 μg solution) with CpG-B and Alum are injected for boosting three times biweekly. Blood samples are obtained from the right ear before each immunization. The sera are stored at −20° C.

Immunoglobulins (IgG/IgA/IgM) in murine sera induced by toxoplasma or parasite/bacterial proteins are assessed in ELISA binding assay (for both original toxoplasma/bacterial antigens and the NR1 subunit of the NMDA receptor) and NMDA receptor internalization assay.

ELISA binding assay: To detect murine IgG/IgA/IgM antibodies specific to toxoplasma/parasite/bacterial proteins and NMDAR-NR1, microtiter plates are coated with toxoplasma/parasite/bacterial proteins or NMDAR-NR1 (100 ng/well). All sera are titrated using Tris buffered saline. The secondary antibody horseradish peroxidase labeled rabbit anti-mouse IgG, IgA or IgM are diluted at 1:5,000 to detect signals.

NMDA receptor internalization assay: NMDA receptor-expressing HEK293 cells in Induction Medium consisting of Neurobasal Medium (Life Technologies Corporation, Carlsbad, CA, USA) with 10% dialyzed FBS, 50 U/mL penicillin-streptomycin, 2.0 g/mL tetracycline, and 0.2 mM memantine are cultured overnight to induce NR1 receptor expression. Cells are harvested and plated at 1.5×10⁵ cells/15 μL in a 96 well plate. Immunized murine sera are diluted and added at a total volume of 50 μL/well. Cells are incubated at 37° C., 5% CO₂ for 20 hours. After incubation, cells are disassociated and transferred to the FACS reading plate (Corning; NY, USA). After washing cells using FACS buffer (2% FBS in PBS), cells are incubated with human Fc receptor binding inhibitor (20 μg/mL; Invitrogen, Carlsbad, CA, USA) and Dead cell stain kit (3:10,000; Invitrogen, Carlsbad, CA, USA) for 15 min on ice. Then, NMDA receptors on cell surface are stained with ART5803 (5.0 μg/mL) or anti-KLH IgG1 (5.0 μg/mL) and Phycoerythrin (PE) conjugated goat anti-human IgG (1:100; Jackson ImmunoResearch Inc, West Grove, PA, USA). The PE signal detection is performed by using FACS Verse (BD Biosciences, Franklin lakes, NJ, USA). FlowJo (BD Biosciences, Franklin lakes, NJ, USA) is used for the analysis of flow cytometry data.

Immunization with toxoplasma and bacterial proteins containing LQNRKLV in mice will result in generating Immunoglobulins (IgG/IgA/IgM) to bind the same toxoplasma/parasite/bacterial antigen proteins. Due to the molecular mimicry between these antigens and NMDA receptor NR1, these antibodies generated in mice by toxoplasma/parasite/bacterial proteins will bind to NR1 subunit of the NMDA receptor in ELISA assay although antibody binding titers in immunized mouse sera will be lower against NR1 subunit of the NMDA receptor compared to original toxoplasma/parasite/bacterial antigens. The immunized mouse sera will induce NMDA receptor internalization in NMDA receptor expressing HEK293 cells.

### Example 3: Characterizing Toxoplasma Reactive T Cells in Patients with Anti-NMDA Receptor Encephalitis and Other Psychotic/Dementia Disorders by ELISpot

Patients with anti-NMDA receptor encephalitis (ANRE), psychosis/dementia conditions including schizophrenia, depression, dementia, and general encephalitis have been shown to have higher titers against T. gondii versus healthy controls. These conditions are referred to as psychosis disorders in subsequent sections concerning the role of T cells in the manifestation of these conditions. Without being bound by theory the link between T. gondii antibodies and psychosis disorders could derive from massive peptide commonalities and molecular mimicry between T. gondii proteins and the NMDA receptor. It is proposed that due to this massive peptide sequence overlap, antibodies generated against the T. gondii proteome during an adaptive host response to infection have the potential to cross-react with the subunits that comprise the NMDA receptor, leading to NMDA receptor dysfunction, and the downstream associated psychosis. In addition to antigen recognition, naïve B-cells generally require helper T-cell support in order to undergo evolution and become antibody secreting plasma cells.

The enzyme-linked immunosorbent spot (ELISpot) assay is performed to detect the presence of cytokine secretion of T lymphocytes. Peripheral blood mononuclear cells (PBMCs) derived from psychosis patients are enriched through ficoll separation and subjected to an ELISpot assay in which they are cultured in the presence of NMDA receptor mimetic T. gondii peptides, in addition to relevant control peptides, in order to determine their overall reactivity to T. gondii antigens. In short, a panel of mimetic peptides are designed to test in an ELISpot assay. These peptides are focused on homologous segments shared between T. gondii and the NMDA receptor NR1 amino terminal domain subunit. Peptides are designed as both HLA I preferred ligands to test CD8 T lymphocyte reactivity, in addition to HLA II preferred ligands to test for CD4 T lymphocyte reactivity. For HLA class I, peptides are designed with 8-10 residues, and for HLA class II, peptides are designed with 13-25 residues in length (Table 4). The peptides are cocultured with PBMCs from patients with ANRE. Interferon gamma (IFNg) and Tumor necrosis factor alpha (TNFa) responses are observed.

If psychosis disorders are mediated through cross-reactivity of T. gondii antibodies towards the NMDA receptor, patients with ANRE will additionally have reactive T cell clones directed towards T. gondii. Therefore, antigen mediated T cell responses will be observed when PBMCs from psychosis patients are cultured in the presence of these peptides, so long as the T. gondii reactive T cells are present in sufficient quantities to meet the sensitivity requirements of the ELISpot assay. These responses will lead to a host of cytokines expressed by the T cells, including IFNg, and TNFa, which will be visualized in the ELISpot system.

TABLE 4
Sequence alignment of NMDA receptor NR1 with proposed T. gondii
peptide HLA ligands for (A) HLA Class I and (B) HLA Class II

| Class | SEQ ID NO | Name | Sequence | Length |
|---|---|---|---|---|
| I | 100 | Toxoplasma-Plus-3b | ---LLQNRKLVAA | 10 |
| | 101 | Toxoplasma-Plus-3a | --DLLQNRKLVA- | 10 |
| | 102 | Toxoplasma-Plus-2 | ---LLQNRKLVA- | 9 |
| | 103 | Toxoplasma-Plus-1b | ----LQNRKLVA- | 8 |
| | 104 | Toxoplasma-Plus-la | ---LLQNRKLV-- | 8 |
| | 105 | NMDAR | MNLQNRKLVQV | 11 |
| | 106 | Toxoplasma-Short | -----LQNRKLV-- | 7 |
| | | | ******* | |
| II | 200 | NMDAR | KFANYSIMNLQNRKLVQVGIYNG-- | 23 |
| | 201 | Toxoplasma-Short-11b | ----------------NRKLV-AALENGKK | 13 |
| | 202 | Toxoplasma-Short-10b | --------------QNRKLV-AALENGKK | 14 |
| | 203 | Toxoplasma-Short-9b | -------------LQNRKLV-AALENGKK | 15 |
| | 204 | Toxoplasma-Short-8b | ------------LLQNRKLV-AALENGK- | 15 |
| | 205 | Toxoplasma-Short-7b | ----------DLLQNRKLV-AALENGKK | 17 |
| | 206 | Toxoplasnna-Short-6b | ---------FDLLQNRKLV-AALENGKK | 18 |
| | 207 | Toxoplasma-Short-5b | --------LFDLLQNRKLV-AALENGKK | 19 |
| | 208 | Toxoplasma-Short-4b | ------KLFDLLQNRKLV-AALENGKK | 20 |
| | 209 | Toxoplasma-Short-3a | ----GKLFDLLQNRKLV-AALENGKK | 21 |
| | 210 | Toxoplasma-Short-2b | --NGKLFDLLQNRKLV-AALENGKK | 22 |
| | 211 | Toxoplasma-Short-lb | -YNGKLFDLLQNRKLV-AALENGKK | 23 |
| | 212 | Toxoplasma-Long | IYNGKLFDLLQNRKLV-AALENGKK | 24 |
| | 213 | Toxoplasma-Short-1a | IYNGKLFDLLQNRKLV-AALENGK- | 23 |
| | 214 | Toxoplasma-Short-2a | IYNGKLFDLLQNRKLV-AALENG-- | 22 |
| | 215 | Toxoplasma-Short-3b | IYNGKLFDLLQNRKLV-AALE------ | 20 |
| | 216 | Toxoplasma-Short-4a | IYNGKLFDLLQNRKLV-AALE------ | 20 |
| | 217 | Toxoplasma-Short-5a | IYNGKLFDLLQNRKLV-AAL------- | 19 |
| | 218 | Toxoplasma-Short-6a | IYNGKLFDLLQNRKLV AA--------- | 18 |
| | 219 | Toxoplasma-Short-7a | IYNGKLFDLLQNRKLV-----------A | 17 |
| | 220 | Toxoplasma-Short-8a | IYNGKLFDLLQNRKLV-------------- | 16 |
| | 221 | Toxoplasma-Short-9A | IYNGKLFDLLQNRKL--------------- | 15 |
| | 222 | Toxoplasma-Short-10a | IYNGKLFDLLQNRK---------------- | 14 |
| | 223 | Toxoplasma-Short-11a | IYNGKLFDLLQNR------------------ | 13 |

### Example 4: Autologous Mixed Lymphocyte Reaction with Pulsed T. gondii Peptides

The mixed lymphocyte reaction (MLR) is a robust reaction that measures cooperation between cells of the immune system. In particular, it involves the co-culture of bone marrow derived dendritic cells with T-cells, with the goal of eliciting an antigen dependent response from the T-cells. Dendritic cells (DCs) are pulsed with peptides of interest that dock onto HLA class II on their surface for presentation to autologous T-cells. T-cells harboring t-cell receptors (TCRs) specific for those HLA/peptide combos are then stimulated through their TCR to expand and proliferate. To measure proliferation of the target cell population, an ELISpot assay (as described above) or flow cytometry using fluorescently labeled HLA peptide pentamers is utilized. The advantage of this technique over ELISpot is that if T-cells targeted towards the antigen of interest are present in low concentrations, the autologous MLR will enable T-cell expansion for downstream analysis.

A ficoll separation of whole blood from patients with ANRE and other psychotic/dementia disorders will be performed to enrich PBMCs. Once PBMCs are enriched, T-cells are isolated using a human T cell enrichment kit, which magnetically separates the T-cells from the rest of the PBMC population. T-cells are then introduced to culture conditions that support their expansion, such as RPMI plus the addition of cytokines such as IL2 and/or IL7. The remaining PBMC fraction is added to a tissue culture treated culture flask in which after two hours, the monocytes adhere, while the remaining cell population remains in suspension. After two hours, the remaining suspension cells are removed, and monocytes go into a 7 day culture supplemented with the cytokines IL4 and GMC-SF to differentiate the monocytes into DCs.

Once T-cells are expanded and autologous DCs are generated, the two cell fractions are subjected to an MLR in which the DCs are pulsed with a panel of T. gondii peptides that have sequence homology with the NR1 subunit of the NMDA receptor. T-cells are taken at different timepoints and analyzed by ELISpot as described herein, in which isolated T cells are stimulated with their respective peptide antigens from the MLR assay, while a fraction is cryopreserved for downstream analysis using flow cytometry.

If T-cell populations exist that are reactive towards HLA/T. gondii peptide combinations, they will be expanded with the help of antigen presenting cells, in this case DCs, displaying a compatible HLA/peptide combination. Once the MLR is completed, a subsequent ELISpot assay is performed to assess whether there is sufficient stimulation of T-cells with their respective peptide species. In addition, fluorescent HLA matched peptide pentamers will be generated and the respective cryopreserved expanded T-cells will be analyzed by flow cytometry to measure the expansion of the target T-cell population.

### Example 5: Characterizing NMDA Receptor Homologous Pathogenic Peptides T Cell Responses in Patients with Anti-NMDA Receptor Encephalitis and Other CNS Disorders

There are several exogenous peptide sequences generated by pathogenic species that share sequence homology with the human NMDA receptor subunit. Without being bound by theory, it is thought that when individuals are infected with these pathogenic species, their immune system generates a functional response in which high affinity antibodies are developed towards these pathogenic species for the purpose of clearing them from the host. Due to the sequence homology between several of these protein species and subunits of the NMDA receptor, it is possible that cross-reactivity of these antibodies towards the NMDA receptor leads to anti-NMDA receptor encephalitis (ANRE) and other psychotic/dementia disorders. As mentioned in example 3, B-cell producing antibodies generally require the assistance of helper cells, generally T cells, in order to class switch into antibody secreting plasma cells.

To characterize the presence of T cells reactive to exogenous, pathogen-produced peptides that are homologous to the NMDA receptor in patients with ANRE and other psychotic/dementia disorders, an ELISpot assay and an autologous MLR assay as described herein are carried forth with the HLA class I and class II compatible peptide sequences.

If psychosis/dementia in particular groups of patients is due to cross-reactivity of antibodies originally developed by the host to target exogenous NMDA receptor homologous peptide sequences, T-cells that are reactive towards pathogenic species will be present. If T-cells are present in high concentrations, simple addition of the peptide to a PBMC mixture from these patients will lead to T-cell activation and will be detected in an ELISpot assay. If T-cells are present in low concentrations, they will be expanded in an autologous mixed lymphocyte reaction and available for characterization in downstream ELISpot assay and flow cytometry assays using fluorescent HLA/peptide pentamers.

### Example 6: Cross-Reactivity of CNS Disorder Patient Antibodies Towards T. gondii and Other Organisms

To determine whether psychiatric or central nervous system diseases or disorders is caused by generation of autoantibodies against the NR1 subunit of NMDA receptor via infection by parasite T. gondii or other organisms which additionally share the common epitope sequence, binding of CNS disorder patient anti-NMDA receptor autoantibody towards recombinant T. gondii proteins, native T. gondii proteins, and T. gondii peptides and other recombinant proteins, native proteins, and peptides derived from organisms in Table 3 will be assessed by ELISA, Western Blot and/or Bio-Layer Interferometry as described herein.

IgG, IgA, and/or IgM monoclonal anti-NMDA receptor autoantibodies derived from patients with CNS disorders (Anti-NMDA Receptor encephalitis, schizophrenia, psychosis, bipolar disorder, depression, epilepsy, dementia, and encephalitis) are assessed for binding to recombinant T. gondii internal kinesin motor domain protein and other organisms' proteins in Table 3 which contains a pathogenic antibody epitope found in the NMDA receptor NR1 subunit (LQNRKLV)

If CNS disorders patient antibodies are reactive towards recombinant proteins in Table 3, there will be cross-reactivity of CNS disorder patient autoantibodies towards these proteins in the binding assays as described herein.

Since recombinant proteins may not accurately mimic native protein conformation, patient-derived monoclonal autoantibodies are assessed for binding to Table 3 organism cell lysate by ELISA or Western Blot.

If CNS disorders patient antibodies are reactive towards native T. gondii proteins, there will be cross-reactivity of CNS disorders patient antibodies towards these proteins in the binding assays as described herein.

As antigen fragments are primarily utilized in initiating immune responses, autoantibodies will be assessed for binding to peptides generated by flanking the epitope sequence with Table 3 organisms surrounding amino acids of varying lengths up to 24 amino acids (an example with T gondii peptides in Table 4).

If CNS disorder patient antibodies are reactive towards T. gondii peptides, there will be cross-reactivity of CNS disorder patient antibodies towards these proteins in the binding assays as described herein.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

1. A method for the treatment or improvement of a psychiatric or central nervous system disease or disorder, wherein the psychiatric or central nervous system disease or disorder is not encephalitis, comprising administering to a subject in need thereof a therapeutically effective amount of an antibody or antigen binding fragment thereof that binds to an N-methyl-D-aspartate (NMDA) receptor comprising a heavy chain variable region and a light chain variable region, the heavy and light chain variable region comprising:
(a) a heavy chain CDR1 as set forth in SEQ ID NO: 11, a heavy chain CDR2 as set forth in SEQ ID NO: 12, a heavy chain CDR3 as set forth in SEQ ID NO: 13, a light chain CDR1 as set forth in SEQ ID NO: 14, a light chain CDR2 as set forth in SEQ ID NO:15, and a light chain CDR3 as set forth in SEQ ID NO:16;
(b) a heavy chain CDR1 as set forth in SEQ ID NO: 17, a heavy chain CDR2 as set forth in SEQ ID NO: 18, a heavy chain CDR3 as set forth in SEQ ID NO: 19, a light chain CDR1 as set forth in SEQ ID NO: 20, a light chain CDR2 as set forth in SEQ ID NO:21, and a light chain CDR3 as set forth in SEQ ID NO:22;
(c) a heavy chain CDR1 as set forth in SEQ ID NO:23, a heavy chain CDR2 as set forth in SEQ ID NO: 24, a heavy chain CDR3 as set forth in SEQ ID NO: 25, a light chain CDR1 as set forth in SEQ ID NO: 26, a light chain CDR2 as set forth in SEQ ID NO:27, and a light chain CDR3 as set forth in SEQ ID NO:28;
(d) a heavy chain CDR1 as set forth in SEQ ID NO: 29, a heavy chain CDR2 as set forth in SEQ ID NO: 30, a heavy chain CDR3 as set forth in SEQ ID NO: 31, a light chain CDR1 as set forth in SEQ ID NO: 32, a light chain CDR2 as set forth in SEQ ID NO:33, and a light chain CDR3 as set forth in SEQ ID NO:34;
or
(e) a heavy chain CDR1 as set forth in SEQ ID NO: 35, a heavy chain CDR2 as set forth in SEQ ID NO: 36, a heavy chain CDR3 as set forth in SEQ ID NO: 37, a light chain CDR1 as set forth in SEQ ID NO: 38, a light chain CDR2 as set forth in SEQ ID NO:39, and a light chain CDR3 as set forth in SEQ ID NO:40.

2. The method of claim 1, wherein the heavy chain variable region comprises an amino acid sequence that has at least 80% sequence identity to any one of SEQ ID NOs: 1, 3, 5, 7, or 9, and the light chain variable region comprises an amino acid sequence that has at least 80% sequence identity to any one of SEQ ID NOs: 2, 4, 6, 8, or 10.

3.-6. (canceled)

7. The method of claim 1, wherein the heavy chain variable region comprises an amino acid sequence identical to any one of SEQ ID NOs: 1, 3, 5, 7, or 9, and the light chain variable region comprises an amino acid sequence identical to any one of SEQ ID NOs: 2, 4, 6, 8, or 10.

8. (canceled)

9. The method of claim 1, wherein the psychiatric or central nervous system disease or disorder is schizophrenia, psychosis, bipolar disorder, depression, epilepsy, or dementia.

10. (canceled)

11. (canceled)

12. The method of claim 1, wherein the antibody or antigen binding fragment thereof binds an (NR1) subunit of the NMDA receptor.

13. The method of claim 1, wherein the antibody or antigen binding fragment thereof comprises a Fab, Fab₂, or an scFv.

14. (canceled)

15. (canceled)

16. A method for identifying an antibody or antigen binding fragment thereof for use in treatment of a psychiatric or central nervous system disease or disorder, the method comprising identifying said antibody or antigen binding fragment thereof that binds specifically to an epitope LQNRKLV (SEQ ID NO: 41) of a NR1 subunit of a NMDA receptor.

17. The method of claim 16, wherein said antibody or antigen binding fragment thereof inhibits binding of an autoantibody that binds to said NMDA receptor.

18. (canceled)

19. (canceled)

20. The method of claim 16, wherein the antibody or antigen binding fragment thereof is human or humanized.

21-27. (canceled)

28. A method for identifying a subject as having a risk of autoantibody associated disease comprising:
(a) obtaining a biological sample from said subject;
(b) assaying said biological sample for antibodies that bind to the amino acid sequence LQNRKLV (SEQ ID NO: 41); and
(c) optionally, outputting a report or identifying said subject as being at high risk or low risk for said autoantibody associated disease.

29. The method of claim 28, wherein said biological sample comprises blood, sweat, saliva, cerebrospinal fluid (CSF), amniotic fluid, or mucus.

30. (canceled)

31. (canceled)

32. The method of claim 28, further comprising treating said subject for said autoantibody associated disease.

33-37. (canceled)

38. The method of claim 32, wherein the antibody or antigen binding fragment thereof binds NMDAR1.

39. (canceled)

40. The method of claim 37, wherein the antibody or antigen binding fragment thereof binds at least one amino acid of the sequence LQNRKLV (SEQ ID NO: 41).

41. The method of claim 28, wherein the autoantibody associated disease comprises a psychiatric or central nervous system disease or disorder.

42. The method of claim 28, wherein the autoantibody associated disease comprises schizophrenia, psychosis, bipolar disorder, depression, epilepsy, or dementia.

43. The method of claim 28, wherein the autoantibody associated disease comprises encephalitis.

44. The method of claim 32, wherein the treating comprises administering a therapeutically effective amount of an antibody that binds the NR1 subunit of NMDAR1.

45. The method of claim 38, wherein the antibody that binds NMDAR1 comprises a heavy chain variable region and a light chain variable region, the heavy and light chain variable region comprising:
(a) a heavy chain CDR1 as set forth in SEQ ID NO: 11, a heavy chain CDR2 as set forth in SEQ ID NO: 12, a heavy chain CDR3 as set forth in SEQ ID NO: 13, a light chain CDR1 as set forth in SEQ ID NO: 14, a light chain CDR2 as set forth in SEQ ID NO:15, and a light chain CDR3 as set forth in SEQ ID NO:16;
(b) a heavy chain CDR1 as set forth in SEQ ID NO: 17, a heavy chain CDR2 as set forth in SEQ ID NO: 18, a heavy chain CDR3 as set forth in SEQ ID NO: 19, a light chain CDR1 as set forth in SEQ ID NO: 20, a light chain CDR2 as set forth in SEQ ID NO:21, and a light chain CDR3 as set forth in SEQ ID NO:22;
(c) a heavy chain CDR1 as set forth in SEQ ID NO:23, a heavy chain CDR2 as set forth in SEQ ID NO: 24, a heavy chain CDR3 as set forth in SEQ ID NO: 25, a light chain CDR1 as set forth in SEQ ID NO: 26, a light chain CDR2 as set forth in SEQ ID NO:27, and a light chain CDR3 as set forth in SEQ ID NO:28;
(d) a heavy chain CDR1 as set forth in SEQ ID NO: 29, a heavy chain CDR2 as set forth in SEQ ID NO: 30, a heavy chain CDR3 as set forth in SEQ ID NO: 31, a light chain CDR1 as set forth in SEQ ID NO: 32, a light chain CDR2 as set forth in SEQ ID NO:33, and a light chain CDR3 as set forth in SEQ ID NO:34; or
(e) a heavy chain CDR1 as set forth in SEQ ID NO: 35, a heavy chain CDR2 as set forth in SEQ ID NO: 36, a heavy chain CDR3 as set forth in SEQ ID NO: 37, a light chain CDR1 as set forth in SEQ ID NO: 38, a light chain CDR2 as set forth in SEQ ID NO:39, and a light chain CDR3 as set forth in SEQ ID NO:40.

46. The method of claim 45, wherein the heavy chain variable region comprises an amino acid sequence that has at least 80% sequence identity to any one of SEQ ID NOs: 1, 3, 5, 7, or 9, and the light chain variable region comprises an amino acid sequence that has at least 80% sequence identity to any one of SEQ ID NOs: 2, 4, 6, 8, or 10.
